(19) United States
(12) Patent Application Publication
(10) Pub. No.: US 2014/0121474 A1
(43) Pub. Date: May 1, 2014

(54) APPARATUS, METHOD, AND COMPUTER-ACCESSIBLE MEDIUM FOR TRANSFORM ANALYSIS OF BIOMEDICAL DATA

(71) Applicant: Edward Ciaccio, Cherry Hill, NJ (US)

(72) Inventor: Edward Ciaccio, Cherry Hill, NJ (US)

(73) Assignee: The Trustees of Columbia University in the City of New York, New York, NY (US)

(21) Appl. No.: 14/114,038

(22) PCT Filed: Apr. 26, 2012
(86) PCT No.: PCT/US12/35154

§ 371 Date: Jan. 8, 2014

Related U.S. Application Data
(60) Provisional application No. 61/479,168, filed on Apr. 26, 2011.

(51) Int. Cl. A61B 1/00; A61B 1/04; A61B 5/044; A61B 5/0205; A61B 5/04; A61B 5/00; A61B 5/046

(52) U.S. Cl. CPC A61B 1/00009; A61B 5/0084; A61B 1/041; A61B 5/046; A61B 5/0205; A61B 5/04012; A61B 5/044; A61B 1/00045
USPC 600/301; 600/300; 600/476; 600/518

(57) ABSTRACT

Exemplary method, computer-readable medium and system can be provided for generating at least one information associated with at least one signal and/or data received from at least one structure. For example, it is possible to determine at least one basis based on a combination of a plurality of portions of the signal(s) and/or the data. It is also possible to generate the information(s) as a function of the basis.

### CROSS-REFERENCE TO RELATED APPLICATIONS

This application claims priority from U.S. Patent Application Ser. No. 61/479,168 filed Apr. 26, 2011, the entire disclosure of which is hereby incorporated herein by reference

### FIELD OF THE DISCLOSURE

The present disclosure relates generally to the analysis of biomedical data, and more specifically, relates to exemplary embodiments of apparatus, method, and computer-readable medium for performing an ensemble transform analysis of biomedical signals.

### BACKGROUND INFORMATION

Representation of independent biophysical sources using Fourier analysis can be inefficient because the basis is typically sinusoidal and general. When complex fractionated atrial electrograms (CFAE) are acquired during atrial fibrillation (AF), the electrogram morphology typically can depend on a mix of distinct non-sinusoidal generators.

Transforms that use a general basis, like Fourier analysis, can be inefficient for representation of independent biophysical sources, or drivers, unless these happen to be generated by sinusoidal functions. In contrast, transforms that use data-driven bases can be efficacious for distinguishing uncorrelated signal components generated by independent drivers, if the morphology is reproduced in the basis. For example, the Fukunaga-Koontz transform can be useful to discern two independent sources in cardiac electrogram data by separating correlated versus uncorrelated components of the variance (e.g., second central moment). (See, e.g., Reference [1]). Development of a data-driven basis and transform that can utilize the ensemble average (e.g., first central moment) may be desirable to detect the actual signal morphologic components originating from distinct sources. This can be useful, for example, in the analysis of complex fractionated atrial electrograms (CFAE), (see, e.g., Reference [2]), which are likely formed by multiple independent generators (e.g., focal areas of high frequency and/or reentrant circuits). (See, e.g., References [3-6]). It is also possible that the ensemble averaging can be done by correlating portions of signals rather than by combining portions of signals by averaging, weighted averaging, or some other statistical function.

Currently, CFAE can be quantified using the dominant frequency (DF), which can be defined as the largest spectral component over the physiologic range of electrical activation rate (e.g., ˜2-10 Hz). (See, e.g., Reference [7]). A calculation of the DF of CFAE using ensemble averaging has typically been done (see, e.g., References [17, 18]). The dominant frequency is typically calculated by bandpass filtering the CFAE, rectification, and low pass filtering of the result, followed by Fourier power spectral analysis. (See, e.g., References [8-9]). However, the filtering process can distort important signal components and the method is typically not robust to phase noise. (See, e.g., References [10-13]). Moreover, signal morphologic components arising from each generator are typically not readily apparent in the sinusoidal basis.

Accordingly, the identification of these generators using efficient methods of representation and comparison may be useful for targeting catheter ablation sites to prevent arrhythmia reinduction. For example, a development of an improved estimate of independent generator frequency and morphologic characteristics may potentially be useful, for example, to target abnormal atrial tissue for catheter ablation (see, e.g., Reference [14]), particularly for persistent AF cases. (See, e.g., References [15, 16]).

Further, certain exemplary embodiments of the present disclosure can be adapted to quantitatively estimate the dominant period (DP, =1/DF) of small intestinal motility from videocapsule image series (see, e.g., Reference [8A]). Celiac disease is typically an autoimmune disease which can manifest as villous atrophy in the small intestinal lining or mucosa (see, e.g., Reference [9A, 10A]). The result can be fissuring of the mucosal surface, as well as a scalloped appearance of the small intestinal mucosal folds, both of which result in an abnormality that is often observable by eye in the acquired videocapsule images. Upon quantitative analysis, it can be shown that the DP of a sequential series of videocapsule images can be significantly longer in celiac disease as compared to control patients, possibly indicating decreased small intestinal motility (see, e.g., Reference [8A]). Furthermore, the relationship between DP and small intestinal transit time can be approximately linear for both celiacs and controls (see, e.g., Reference [8A]). Thus, frequency analysis using videocapsule image frames can be potentially useful for clinical diagnostics.

Accordingly, there can be a need to address and/or overcome at least some of the above described deficiencies and issues.

### SUMMARY OF EXEMPLARY EMBODIMENTS

These and other deficiencies can be addressed with the exemplary embodiments of the present disclosure.

For example, according to certain exemplary embodiments of the present disclosure, apparatus, methods, and computer-readable medium can be provided for analyzing biomedical data using a new transform which does not distort analyzed signals and can be robust to phase noise, for calculation of the DF, and identification of independent generator frequency and morphology in CFAE. Exemplary derivations of the exemplary transform procedure according to certain exemplary embodiments of the present disclosure can also be implemented. Exemplary embodiments of the present disclosure can also provide comparisons of the exemplary transform to Fourier analysis to measure the DF of CFAE, and the robustness of each method of DF measurement when random noise is added to the signal. Additionally, the frequencies of simulated drivers embedded in CFAE in the presence of phase noise and interference can be detected with each exemplary procedure. Further, correspondence(s) can be shown between basis vectors of highest power derived from the new transform, versus actual CFAE morphology and synthesized drivers.

According to further exemplary embodiments of the present disclosure, apparatus, method, and computer-readable medium can be provided for an evaluation of CFAE signals. For example, the ensemble average of signal segments can be used to construct a data-driven basis, and it can be shown to have significant advantages over Fourier analysis for correct prediction of the DF of independent drivers in presence of phase noise and interference, as well as for representation of CFAE signals in general, and the distinctive morphologic components associated with each independent synthetic driver that can be tested. The exemplary transform can have possible applications for targeting drivers of atrial fibrillation during clinical catheter ablation to prevent reinduction of the arrhythmia, as well as for improved understanding of the mechanisms by which paroxysmal and persistent AF can be initiated and maintained.

According to additional exemplary embodiments of the present disclosure, synthetic image sequences can be generated with spatiotemporal phase noise, random noise, and air bubbles imposed, to validate the measurement of the DP in videocapsule image series. Instead of using average image brightness level for spectral analysis, the image frames can be analyzed pixel-by-pixel, which can increase robustness to presence of extraneous features and noise. Because of the smoothing effect of analyzing the spectra from many pixels and taking the mean, the repetition rate of a synthesized sequence of images can be detectable even at high noise level.

According to yet further exemplary embodiments of the present disclosure, apparatus, methods, and computer readable medium can be provided for a robust spectral analysis of videocapsule images of celiac diseases. For example, videocapsule endoscopy can be useful to detect mechanical rhythms of the small intestinal lumen via the dominant period (DP) spectral calculation. However, noise and air bubbles obscure image features and can mask rhythms. Fourier versus ensemble averaging spectral analysis can be used to detect simulated periodicity in small intestinal images. According to certain exemplary embodiments, for example, ten-to-twenty sequential image frames sampled at, for example, 2 frames/second can be extracted from each of 10 videoclips obtained from 10 celiac disease patients (e.g., 576×576 pixel resolution). These frames can be repeated to create a synthesized sequence about 200 frames in length, typical for quantitative analysis of videoclips. Random noise, spatiotemporal phase shift, and imposition of air bubble frames can be used for sequence degradation. Power spectra can be then computed pixel-by-pixel from the brightness levels over 200 image frames.

The tallest peak in the mean power spectrum calculated from the 576×576 pixel-level spectra can be taken as the DP. The absolute difference between the actual DP based on repetition of the frames sequence, versus the estimated value from spectral analysis, can be tabulated, as can the speed of computation for Fourier versus ensemble averaging methods. For the additive noise levels, e.g., the mean absolute difference between estimated versus actual DP can be, for example, 0.0547±0.0688 Hz for Fourier versus 0.0031±0.0127 Hz for ensemble (e.g., p<0.001 in mean and standard deviation). The mean time for computing 331,776 pixel spectra per videoclip can be, for example, 12.31±0.01 s for Fourier versus 4.86±0.01 s for ensemble (p<0.001). Ensemble spectral analysis according to certain exemplary embodiments of the present disclosure can be robust to additive noise and spatiotemporal jitter, and useful for rapid DP calculation in videocapsule image series.

According to further exemplary embodiments of the present disclosure, method, computer-readable medium and system can be provided for generating at least one information associated with at least one signal and/or data received from at least one structure. For example, it is possible to determine at least one basis based on a combination of a plurality of portions of the signal(s) and/or the data. It is also possible to generate the information(s) as a function of the basis.

In one exemplary embodiment, the combination can include a summation, an average, a weighted average and/or a statistical representation. The summation can include a summation of a plurality of segments of the signal(s) or the data. The generation of the information can include an application of a transform relating the combination to at least one frequency of the signal(s) so as to generate a power spectrum. The signal(s) or the data can include a video-capsule image associated with one of a celiac disease or a cardiac signal as obtained during atrial fibrillation. The information can include a dominant frequency, a dominant period, a mean, and/or a standard deviation in a power spectral profile.

It is possible to qualify at least one characteristic associated with the signal(s) or the data based on the transform, a noise, an interference and/or an artifact in generating a reconstruction of the signal(s) based on the transform. It is also possible to increase a frequency resolution for a given time period of the signal(s) or the data based on the transform. Further, it is possible to cause a recognition of a source pattern of the signal(s) or the data based on the transform. The signal(s) or the data can be an image.

These and other objects, features and advantages of the present disclosure will become apparent upon reading the following detailed description of exemplary embodiments of the present disclosure, when taken in conjunction with the appended drawings and claims.

### BRIEF DESCRIPTION OF THE DRAWINGS

Further objects, features and advantages of the present disclosure will become apparent from the following detailed description taken in conjunction with the accompanying Figures showing illustrative embodiments of the present disclosure, in which:

FIGS. 1A-1D are graphs of exemplary power spectrum constructions according to certain exemplary embodiments of the present disclosure;

FIGS. 2A-2D are graphs of exemplary synthetic drivers used for an exemplary spectral analysis and reconstruction according to certain exemplary embodiments of the present disclosure;

FIGS. 3A-3D are graphs of exemplary average spectra for the exemplary simulated drivers according to certain exemplary embodiments of the present invention;

FIGS. 4A-4D are graphs of exemplary normalized inner product for 481 basis vectors with magnitude 0 at the bottom right and magnitude 1 at top according to certain exemplary embodiments of the present disclosure;

FIG. 5A is a graph of an exemplary Fourier spectrum;

FIG. 5B is a graph of an exemplary ensemble spectrum according to certain exemplary embodiments of the present disclosure;

FIGS. 6A-6D are graphs of exemplary ensemble basis vectors constructed from a synthesized signal according to certain exemplary embodiments of the present disclosure;

FIG. 7A is a graph of exemplary CFAE from a paroxysmal AF patient;

FIG. 7B is a graph of exemplary CFAE with random noise;

FIGS. 7C and 7D are graphs of Fourier power spectrums for the signals shown in FIGS. 7A and 7B, respectively;

FIGS. 7E and 7F are graphs of exemplary ensemble power spectrums for the signals shown in FIGS. 7A and 7B, respectively, according to certain exemplary embodiments of the present disclosure;

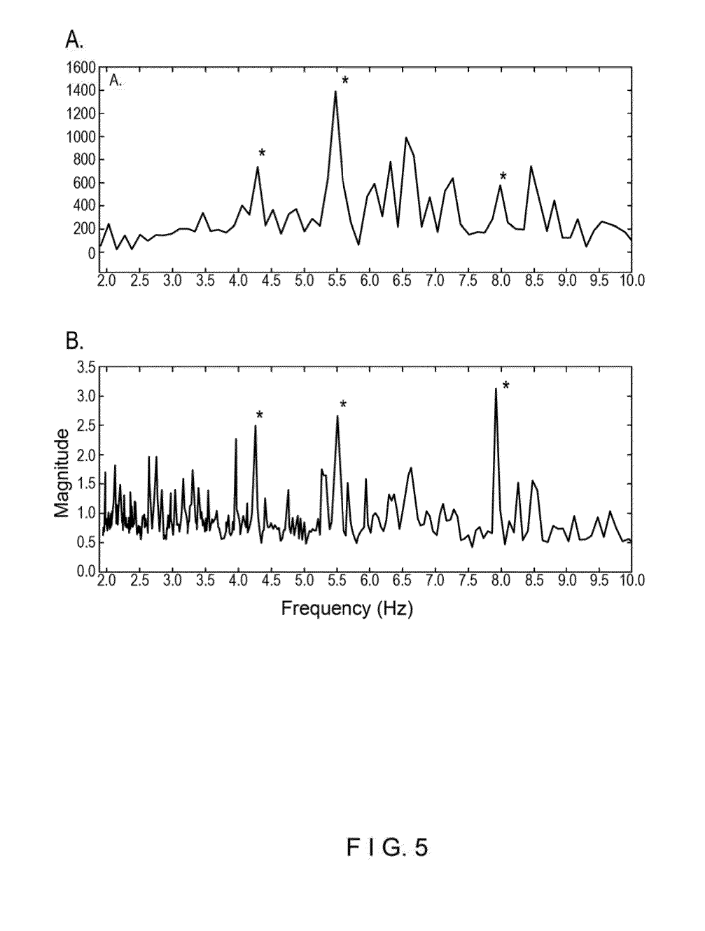

FIGS. 5A and 8B are graphs showing exemplary CFAE reconstructions with 1 and with 10 basis vectors using Fourier analysis;

FIGS. 8C and 8D are graphs showing exemplary CFAE reconstructions with 1 and with 10 basis vectors using ensemble analysis according to certain exemplary embodiments of the present disclosure;

FIGS. 9A-9C are graphs showing exemplary statistics of Fourier and ensemble average reconstruction error for real CFAE signals;

FIG. 10A is a graph of an exemplary effect of a transient on a CFAE signal;

FIG. 10B is a graph of an exemplary CFAE from left inferior pulmonary vein of a persistent AF patient;

FIG. 10C is a graph of an exemplary Fourier power spectrum of the signal shown in FIG. 10B;

FIG. 10D is a graph of an exemplary ensemble average power spectrum of the signal shown in FIG. 10B according to certain exemplary embodiments of the present disclosure;

FIGS. 11A-11C are graphs of additional exemplary Fourier versus ensemble averaging power spectra with the transient added to CFAE signals;

FIGS. 12A-12F are exemplary videocapsule series images;

FIGS. 13A-13F are exemplary videocapsule series images;

FIGS. 14A-14D are graphs of exemplary Fourier power spectra;

FIGS. 15A-15D are graphs of exemplary ensemble power spectra according to certain exemplary embodiments of the present disclosure;

FIGS. 16A-16D are graphs of exemplary Fourier power spectra;

FIGS. 17A-17D are graphs of exemplary ensemble power spectra according to certain exemplary embodiments of the present disclosure;

FIG. 18 is an illustration of an exemplary block diagram of an exemplary system in accordance with certain exemplary embodiments of the present disclosure;

FIGS. 19A-19D are exemplary graphs of frequency spectra used for analysis for CFAE with two closely spaced frequency components at the low end of range A;

FIGS. 20A-20D are exemplary graphs of frequency spectra used for analysis of CFAE with synthetic frequency components spaced further apart than that of FIG. 19;

FIGS. 21A-21F are exemplary graphs of atrial electrograms used as patterns A and B to be detected in the set of 216 initial recording sequences;

FIGS. 22A-22D are exemplary graphs of transform coefficients when two patterns A and B are embedded in interference;

FIGS. 23A-23D are exemplary graphs of spectral signatures of pattern A and B computed from the basis vectors derived from the mean signal;

FIGS. 24A-24D are exemplary graphs of a Euclidean distance between the power spectrum of the mean from 216 recordings, and the spectral signatures of 214 individual recordings with interference added; and

FIG. 25 is a flow chart illustrating a method in accordance with an exemplary embodiment of the present application.

Throughout the drawings, the same reference numerals and characters, unless otherwise stated, are used to denote like features, elements, components, or portions of the illustrated embodiments. Moreover, while the present disclosure will now be described in detail with reference to the figures, it is done so in connection with the illustrative embodiments and is not limited by the particular embodiments illustrated in the figures.

### DETAILED DESCRIPTION OF EXEMPLARY EMBODIMENTS

### Exemplary Methods

FIG. 25 shows a flow chart illustrating use of a method in accordance with an exemplary embodiment of the present application.

As shown in FIG. 25, in procedure 2501, a signal can be received. The signal may be (i) a CFAE signal, (ii) an image received from a video capsule camera, (iii) a biomedical signal, and/or (iv) any form of data. In procedure 2502, a combination of a plurality of portions of the signal can be taken. The combination may be a summation, an average, a weighted average, and/or any other statistical measurement. In procedure 2503, data driven basis vectors can be determined from the combination of the plurality of portions of the signal. In procedure 2504, information can be generated as a function of, or based on, the basis vectors determined from procedure 2503. Further, in procedure 2505, the information generated can be analyzed and may be used in biomedical procedures, although not limited thereto. Any of the exemplary procedures set forth above can be performed by the exemplary system of FIG. 18 described herein.

### Exemplary Transform Equations

The autocorrelation coefficient r_{φ} at lag φ can be given by the inner product of two mean-zero signal vectors, e.g.:

r_{φ}=1/Nx₀^{T}·x_{φ} (1)

where x0 and x_{φ} can be of length N and can be given by, e.g.:

x₀=[x(k)x(k+1) . . . x(k+N−1)]^{T} (2a)

x_{φ}=[x(k−φ))x(k−φ+1) . . . x(k−φ+N−1)]^{T} (2b)

and the vectors can be normalized a priori by scaling to unity variance. Suppose that lag φ represents a segment of x0 that is w sample points long. Exemplary Eq. (1) can then be rewritten as, e.g.:

r_{w}=1/nwΣᵢs_{wi}^{T}·s_{wi+1} i=1,n (3)

where s_{w} can be segments of signal x₀ having length w, e.g.:

s_{wi}=[x(w·i+1),x(w·i+2), . . . x(w·i+w)]^{T} (4a)

s_{wi+1}=[x(w·(i+1)+1),x(w·(i+1)+2), . . . x(w(i+1)+w)]^{T} (4b)

and the number of signal segments can be, e.g.:

n=int(N/w) (5)

Based on these exemplary equations, the autocorrelation function for w can be described as a graph of the mean autocorrelation between successive signal segment pairs swi, swi+1 as given by exemplary Eq. (3), versus segment length w. The segment length can be converted to a frequency, e.g.:

f=sample rate/w (6)

which can reduce to 1/w when the sample rate is, for example, 1 kHz and the time units are milliseconds. The peak in the autocorrelation function over, for example, a frequency range f1 to f2 (1/w1 to 1/w2) that is physiologic for electrical activation rate has been used to estimate the DF in atrial electrograms. (See, e.g., References [19-21]).

A more robust alternative for adapting the autocorrelation function to spectral analysis can use ensemble averaging. (See, e.g., References [17, 18]). The ensemble average vector e_{w} can be obtained by averaging the n successive mean zero segments of signal x, each segment being, for example, of length w, e.g.:

e_{w}=1/n·U_{w}·x (7a)

U_{w}=[I_{w}I_{w} . . . I_{w}] (7b)

where I_{w} can be w×w identity submatrices used to form the signal segments that can be extracted from x and summed. Thus, e.g.:

e_{w}1/nΣᵢs_{wi} i=1,n (8)

where s_{wi} can be as given in exemplary Eq. (4a). The power in the ensemble average can be described by, e.g.:

$$\begin{matrix}{P}_{w} = 1 / w {\underline{e}}_{w}^{T} \cdot {\underline{e}}_{w} & ( 9 a ) \\ = 1 / {n}^{2} w {\underline{x}}^{T} {U}_{w}^{T} {U}_{w} \underline{x} & ( 9 b ) \\ = 1 / {n}^{2} w {\sum }_{i} {\sum }_{j} {\underline{s}}_{wi}^{T} {\underline{s}}_{wj} & ( 9 c )\end{matrix}$$

where exemplary Eq. (9b) and (9c) can be formed by substituting exemplary Eq.'s (7) and (8) into exemplary Eq. (9a), and i and j can be segment numbers from 1 to n. Exemplary Eq. (9c) can be similar to exemplary Eq. (3), except that instead of computing the autocorrelation between successive signal segment pairs s_{wi}, s_{wi+1} only (lag w), it can be computed between signal segments s_{wi}, s_{wj}. P_{w} can be therefore equivalent to computing the mean autocorrelation coefficient from n points in the autocorrelation function separated by lag w, e.g., to averaging the autocorrelation coefficients at lags w, 2w, 3w, nw. However, to generate P_{w}, in this way rather than by using exemplary Eq. (9c) would typically require a sequence length 2N to convolve the signal with itself along its entire length, halving the time resolution and doubling the sequence length generally needed for analysis.

To generate an ensemble power spectrum, the root mean square (RMS) power has been used (see, e.g., References [17, 18]), e.g.:

P_{wRMS}=√(P_{w}) (10)

where sqrt can be the square root function and the units can be millivolts. The power spectrum can be displayed by plotting sqrt(n)·P_{RMS} versus frequency f as computed from exemplary Eq. (6). The sqrt(n) term can level the spectral baseline, which would otherwise decrease by 1/sqrt(n), the amount of noise falloff per number of summations n used for ensemble averaging. From exemplary Eq.'s 5, 9c, and 10, the displayed RMS power can be written as, e.g.:

√[n·P_{wRMS]=√[}1/NΣᵢΣⱼs_{wi}^{T}s_{wj}] (11)

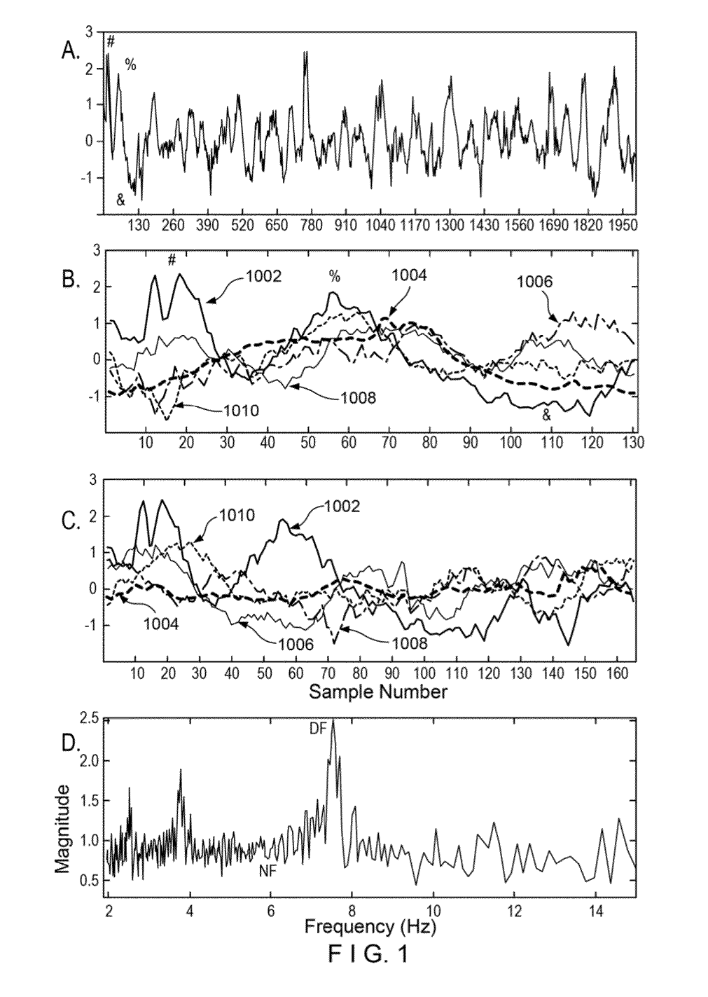

An example of ensemble average power spectrum construction is shown, for example, in graphs of FIGS. 1A-1D. A typical CFAE from the left inferior pulmonary vein ostia during longstanding persistent AF is shown in panel A. The summing of the first four segments of width w=130 is shown in panel B, represented as 1002, 1010, 1008, and 1006, respectively. Some corresponding features between the CFAE trace in panel A and the first segment in panel B are noted (labeled #, %, &). The segmented traces often have peaks at similar locations (panel B). The ensemble average for the segments of width w=130 is shown as a dashed trace 1004 (panel B). It has similarities to segments 1-4 shown, and to many other CFAE segments having width w=130 (from Eq. (5), int(8192/130)=63 segments in total). For perspective, the x-axis scale can be marked in intervals of 130 (panel A) with each scale mark representing the start of a new segment number. In panel C, segments with width w=165 sample points are shown. The peaks may not be well-aligned, and the ensemble average, again shown as a dashed blue line, is of a lower amplitude than in panel B. Thus segments with width w=165 may not be well correlated.

The ensemble average calculation can be repeated for all segments w in the frequency range of interest, as given by exemplary Eq. (6) with a sampling rate of 977 Hz. The RMS power in the ensemble average was then plotted using exemplary Eq. (11) (FIG. 1D). The DF occurs, for example, at 7.52 Hz, corresponding to w=130 sample points (panel B). In contrast, the noise floor marked at ‘NF’, with f=5.92 Hz, occurs, for example, at w=165 sample points (panel C). The ensemble averaging spectrum thus can display correlated components as higher power, and can have more detail at the lower spectral range due to the w=1/f relationship (exemplary Eq. (6)). In addition, DF subharmonics can be pronounced.

The relation between the ensemble power spectrum and the Fourier power spectrum can be described as follows. Based upon the Wiener-Khinchin theorem, the Fourier transform of the autocorrelation function of a signal can be the power spectrum of that signal, e.g.:

$$\begin{matrix}S ( f ) = {\sum }_{φ} {r}_{xx} ( φ ) {e}^{- j2 π f φ} d φ & ( 12 a ) \\ = 1 / nw {\sum }_{φ} {\underline{x}}_{0}^{T} {\underline{x}}_{φ} {e}^{- j2π f φ} & ( 12 b ) \\ = 1 / N {\sum }_{i} {\sum }_{w} ( {\underline{s}}_{wi}^{T} {\underline{s}}_{wi + 1} ) {e}^{- j2 π f w} & ( 12 c )\end{matrix}$$

where S can be the power spectral density, dφ can be the phase lag w, i can be the segment number, and substitution using exemplary Eq.'s (1) and (3) can be utilized to form exemplary Eq.'s (12b) and (12c). The Fourier power spectral density calculation can decompose the autocorrelation function into its native sinusoids. Therefore, in contrast to autocorrelation spectral analysis Eq. (3), both ensemble and Fourier spectral analyses can account for periodicity at lags—ensemble by averaging Eq. (9c) and Fourier by fitting sinusoids Eq. (12c).

The ensemble average of segments having length w can be a representation of correlated signal components at the corresponding frequency (e.g., =sample rate/w), and can be potentially useful for signal reconstruction. From exemplary Eq.'s (7b) and (9b), an ensemble average transformation matrix can be described as, e.g.:

$$\begin{matrix}{T}_{w} = {U}_{w}^{T} {U}_{w} & ( 13 a ) \\ = [ \begin{matrix}{I}_{w} & {I}_{w} & … & {I}_{w} \\ {I}_{w} & {I}_{w} & … & {I}_{w} \\ & & … & \\ {I}_{w} & {I}_{w} & … & {I}_{w}\end{matrix} ] & ( 13 b )\end{matrix}$$

Signal x can then be decomposed using the linear transformation, e.g.:

a_{w}=1/nT_{w}·x (14)

where a_{w} can be basis vectors, n can be as given in exemplary Eq. (5), and a_{w} and w are N×1 in dimension. Column-wise, each identity submatrix in exemplary Eq. (13b) can serve to extract and sum one segment of w sample points in x (e.g., exemplary Eq. (14)), with the sum total being projected, for example onto the canonical basis. Row-wise the identity matrices can serve to repeat the ensemble average of length w over a total length N during construction of a_{w}. Thus, the transformation matrix of exemplary Eq. (13) can act to decompose signals into periodic ensemble averages. Using the resulting basis vectors, signal x can be projected into ensemble space, e.g.:

x^{T}·a_{w}=1/n²wx^{T}·T_{w}·x=P_{w} (15)

where the middle and RHS in exemplary Eq. (15) can be obtained by substitution and rearrangement using, for example, exemplary Eq.'s (9), (13) and (14). Exemplary Eq. (15) states, for example, that if each signal segment of length w is correlated with the ensemble average at w (LHS), the resulting correlation coefficient equals the ensemble average power (RHS).

In the case when N≠n·w above, the transformation matrix T_{w} (e.g., exemplary Eq. (13b)) is preferably padded by N−(n·w) rows and columns, for example, by adding 0's as elements at the matrix's right edge, and adding clipped identity matrices as elements at bottom edge so the overall dimension is N×N. T_{w} can be singular for all w, since two or more rows and two or more columns are typically identical, e.g., it typically has no inverse. Thus, it is generally not possible to transform any particular basis vector a_{w} back to x, as can be intuitively obvious—an ensemble average, for example, cannot be transformed back into its original signal. Suppose now that multiple transformation equations i=1, γ are summed, for example, e.g.:

$$\begin{matrix}{\underline{a}}_{w 1} + … + {\underline{a}}_{w γ} = 1 / {n}_{1} {T}_{w 1} \cdot \underline{x} + … + 1 / {n}_{γ} {T}_{w γ} \cdot \underline{x} & ( 16 a ) \\ = [ {\sum }_{i} ( 1 / {n}_{i} {T}_{wi} ) ] \cdot \underline{x} & ( 16 b )\end{matrix}$$

This can be rewritten, e.g.:

Σᵢa_{wi}=v= [symbol] x (17a)

[symbol]

=Σᵢ(1/nᵢT_{wi}) (17b)

where v can be the estimate of x and [symbol] can be the total transform matrix. Any two basis vectors aᵢ and aⱼ i≠j, used for construction of v, will typically be orthogonal since they can be formed from vectors in Ti versus Tj that can be orthogonal, except when i/j is reducible to a small integer ratio. An example of a total transform matrix constructed from Ti and Tj, with dimension N=6, can be, for example:

$$\begin{matrix}= 1 / 3 {T}_{2} + 1 / 2 {T}_{3} \\ = [ \begin{matrix}.83 & 0 & .33 & .5 & .33 & 0 \\ 0 & .83 & 0 & .33 & .5 & .33 \\ .33 & 0 & .83 & 0 & .33 & .5 \\ .5 & .33 & 0 & .83 & 0 & .33 \\ .33 & .5 & .33 & 0 & .83 & 0 \\ 0 & .33 & .5 & .33 & 0 & .83\end{matrix} ]\end{matrix} \qquad ( 18 )$$

The magnitudes are typically greatest along the main diagonal and equal Σ1/nᵢ, where n can be given by exemplary Eq. (5). This matrix is typically not invertible (e.g., Matlab ver. 7.7, R2008b). In general, as with the individual transform matrices, the total transform matrix will typically not be invertible.

Consider how [symbol] acts to transform signal x. Let a subset γ of highest basis vectors, when ranked in descending order of power, be formed from [symbol] (see, e.g., exemplary Eq. (17)). In this case [symbol] can transfer the most correlated periodic components of the signal to form estimate ν. The relative amplitude relationships of these correlated components, each extracted by a different [symbol] embedded in [symbol] , can be maintained by scale factor 1/nᵢ during transformation (see, e.g., exemplary Eq. (16)). However, as each correlated component is typically independent (e.g., no harmonic relationships), their combination can cause the ‘noise’ power in v to increase by √γ. To maintain the same power for best match with x, the estimate can, for example, either be scaled by 1/√γ, or alternatively, v and x can be scaled to the same power. Any unique signal structure that is not periodic can also be transformed by T, but it is typically via the main diagonal, for example, and not the off-diagonal elements (e.g., which sum and reinforce correlated content only). As γ is increased, the magnitude of the main diagonal elements can increase so that T can act in part as an N×N identity matrix I_{N} to directly transfer the unique uncorrelated detail during formation of v. So long as ai and aj are approximately orthogonal, the unique detail, as well as correlated components, can maintain their correct amplitude relationships in v, since they can be added in tandem and scaled by 1/nᵢ.

### Exemplary Atrial Electrogram Clinical Data

Exemplary clinical data was collected implementing/utilizing certain exemplary embodiments of the present disclosure. For example, atrial electrograms can be recorded in a series of 20 patients, 10 with paroxysmal and 10 with longstanding persistent type, referred to the Columbia University Medical Center cardiac electrophysiology (EP) laboratory for catheter ablation. Two bipolar recordings of at least 10-second duration can be obtained from six anatomical regions: the ostia of the left superior and inferior pulmonary veins (LSPV, LIPV), the ostia of the right superior and inferior pulmonary veins (RSPV, RIPV), and the anterior and posterior left atrial free wall (ANT, POS). The recordings can be obtained from these regions via the distal bipolar catheter ablation electrode during sustained AF prior to any ablation. Using standard settings, the signals can be filtered in hardware at acquisition to remove baseline drift and high frequency noise (first order filter pass band: 30-500 Hz). In each patient, for example, a CFAE sequence 8192 sample points long (e.g., ˜0.8 seconds) as determined visually by two clinical electrophysiologists can be retrospectively selected for analysis from two sites at each of the six locations. CFAE can be defined, for example, as atrial electrograms with three or more deflections on both sides of the isoelectric line, or continuous electrical activity with no well-defined isoelectric line (see, e.g., Reference [2]). In all, for example, 216 of 240 recordings met these criteria, as determined by two cardiac electrophysiologists, and can be used for the exemplary further analysis. For example, no ventricular component, corresponding to the QRS deflection of the electrocardiogram, was visually evident in the CFAE. In these bipolar recordings it is typically uncommon for QRS artifact to be evident in CFAE obtained from the pulmonary veins and free wall. The signals were sampled, for example, at 0.98 kHz, and stored in both raw form, and after normalization to mean zero and unity variance.

### Exemplary Tests of Fourier Versus Exemplary Ensemble Procedures

The following exemplary tests can illustrate the efficacy of the new exemplary transform versus Fourier analysis for representation of frequency and morphologic components of, for example, CFAE. The Fourier DF method can be optimized, for example, when CFAE recordings are bipolar and approximately 8 s in length (see, e.g., References [11, 22-23]). Accordingly, these can be used in the exemplary tests. The 8 s sequences were readily available from retrospective data, since, for example, during electroanatomic mapping, recordings with short sequence length are commonly acquired from each site to minimize the procedure time.

### Exemplary Orthogonality of the Ensemble Basis

The inner product of ensemble basis vectors can be determined (e.g., using a computer arrangement) as, e.g.:

dpᵢⱼ=a_{wi}^{T}·a_{wj} (19)

for all pairs i, j from w=500 to w=20 (f=2-50 Hz) for one paroxysmal and one persistent CFAE signal. The dp's can be graphed i versus j. The ensemble basis can be considered to be orthogonal if dp=1.0, i=j, and dp≈0, i≠j, except for small integer relationships in i/j. For comparison, dp can also be calculated with the Fourier basis using the same paroxysmal CFAE signal.
Exemplary Spectral Analysis of Synthetic Drivers with Phase Noise and Interference

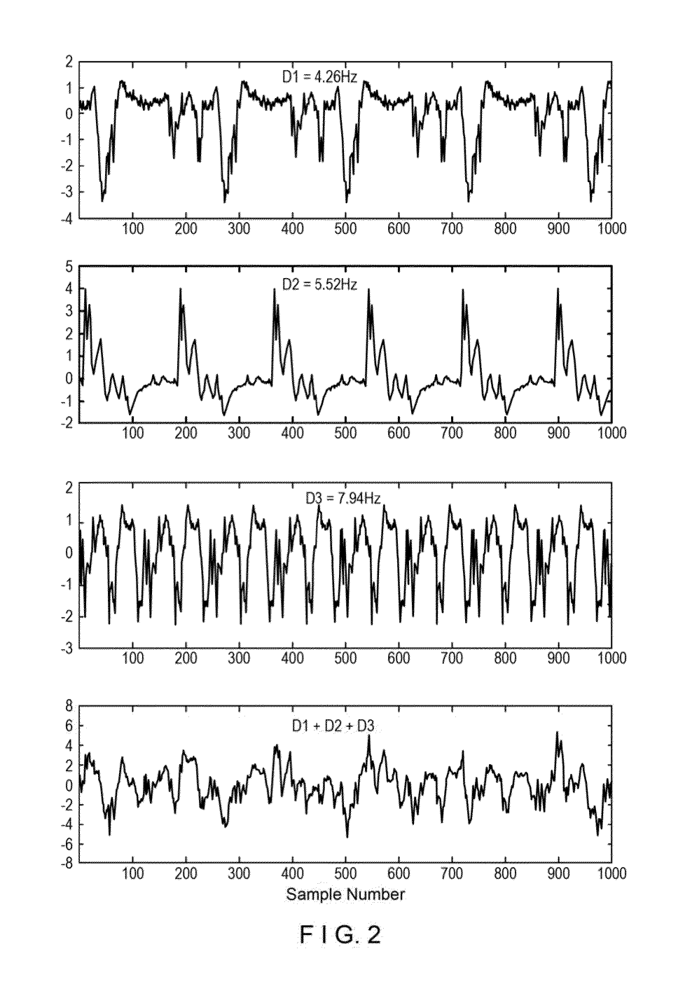

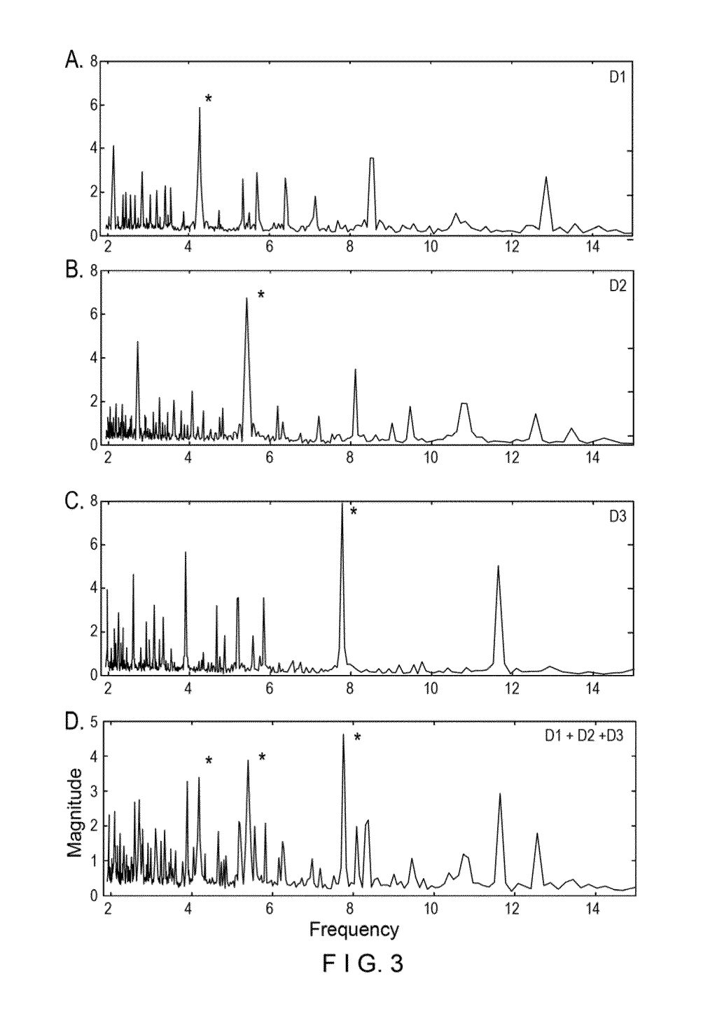

A number of, e.g., three simulated independent drivers with unrelated fundamental, or dominant frequencies (DF), can be constructed, for example, from distinct CFAE deflections extracted from a single recording in one paroxysmal AF patient. The sequence lengths can be, for example, 229 ms, 177 ms, and 123 ms to simulate independent drivers D1, D2, and D3 with DF of 4.37 Hz, 5.65 Hz, and 8.13 Hz, respectively. The simulated independent generator frequencies were within the typical range of DFs that are observed in CFAE. (See, e.g., References [2,4,7]). These can be normalized to mean zero and repeated to, for example, 8192 sample points. As shown in FIG. 2, D1 can include primarily downward deflections, D2 can include upward deflections, and D3 can be biphasic. Their combination is shown, for example, in FIG. 2D. The ensemble average spectra for these simulated drivers and for their sum is shown, for example, in corresponding panels of FIG. 3, with DFs marked by asterisks. The harmonics of each simulated generator may not overlap. It is also possible that averaging segments of the ensemble average can be used to remove harmonic interaction and reduce spectral cross terms.

Phase noise can then be added by randomly and independently shifting the timing of each driver pulse (each 229, 177, or 123 ms interval) using a mean zero random number generator with standard deviation of ±16 ms. Interference can be added by summing the resulting synthetic signal D1+D2+D3 with one of 216 scaled CFAE signals (e.g., the CFAE signals themselves acted as interference for measurement of the synthetic driver characteristics). The following combinations of gains for the phase noise random vector (p) and interference (i) can be used for assessment, for example: (p=1×, i=1×), (p=0.5×, i=2×), (p=0.3×, i=3×), and (p=0×, i=±1× . . . ±10×). Fourier and ensemble power spectra can be constructed in the range 2-10 Hz from the resulting signals. The spectral peaks can be ranked by amplitude, and the sum of ranks for peaks having frequencies of 4.26 Hz, 5.52 Hz, and 7.94 Hz, with a tolerance of ±0.2 Hz, can be tabulated. The best (e.g., minimum) sum of ranks is, for example, 6 which can occur when the driver frequencies at 4.26 Hz, 5.52 Hz, and 7.94 Hz are ranked, for example, 1^{st}, 2^{nd}, and 3^{rd} in amplitude, in certain combination, among all spectral peaks.

### Exemplary Identification of Synthetic Driver Morphology

As the exemplary ensemble procedure, but aside from the Fourier transform, typically has a data-driven basis, only ensemble was used in this exemplary test. The synthetic drivers with additive phase noise and interference described in exemplary Test 2 can be corrupted using two noise gain sets, for example: p=0.3×, i=3×, and p=0×, i=5×, where the interferences i can include the 216 CFAE signals (e.g., 216 comparisons for each of the two noise gain sets). The mean squared error difference between each original synthetic driver without noise (FIG. 2), versus the corresponding ensemble basis vector of the corrupted signal at 123 ms, 177 ms, and 229 ms, the periods of the drivers, when both were normalized to unity power, can be tabulated in mV²/ms.

Exemplary Degradation of DF in CFAE with Additive Random Noise

This exemplary test can be used to determined the efficacy of each transform to detect the DF of CFAE in presence of random noise (no added synthetic drivers). For each of 20 selected CFAE with a prominent DF (sharp peak and low noise floor), random white noise can be added, for example, with a standard deviation of 0.16 mV, approximately half that of the raw CFAE signals. The DF of the resulting CFAE signal with additive random noise can be determined. The absolute difference in DF before versus after random noise addition can be tabulated. This exemplary procedure can be repeated, for example, for 10 different additions of random noise. The mean and standard deviation in the absolute difference in DF before versus after addition of random noise can be calculated for ensemble versus Fourier spectral analysis. The entire process can be repeated for random white noise with a standard deviation of 0.32 mV, approximately equal to the standard deviation of the raw CFAE signals.

### Exemplary CFAE Reconstruction

The exemplary 216 CFAE recordings (e.g., no added synthetic drivers) can be each decomposed and then reconstructed using 1-12 Fourier or ensemble basis vectors. The mean squared error difference between each CFAE and its reconstruction from the ordered bases can be determined. The reconstructions used can be, for example, e.g.:

$$\begin{matrix}\begin{matrix}{\underline{v}}_{1} = {\underline{a}}_{w 1} {\underline{v}}_{2} \\ = {\underline{a}}_{w 1} + {\underline{a}}_{w 2}\end{matrix} \\ ⋮ \\ {\underline{v}}_{12} = {\underline{a}}_{w 1} + {\underline{a}}_{w 2} + … + {\underline{a}}_{w 12}\end{matrix} \qquad ( 19 )$$

where a_{w1} to a_{w12} can be, for example, the top 12 basis vectors in descending order of power. The average error can be determined for Fourier versus ensemble reconstruction.

### Exemplary Single Driver Test

The CFAE signals can be then altered or modified by adding a low-power transient component at, for example, 200 sample point intervals (e.g., 977 samples per second/200 samples ˜5 Hz). The transient itself can include a 42 sample point long biphasic component extracted from a CFAE acquired from the LSPV ostia during persistent AF. This transient can have properties of mean=0.13 mV, standard deviation=0.54 mV, and peak-peak values of ˜±1 mV. CFAEs after addition of the low-power transient can be analyzed using ensemble and Fourier spectral analysis to determine whether the component can be readily identified. Identification can be defined to be presence of a distinct power spectral peak, with the base of the peak reaching the surrounding noise floor.

For the exemplary tests described above, the ensemble averaging power spectrum was generated as described by exemplary Eq.'s 9-11 and the accompanying text. Exemplary Fortran code used for ensemble spectra calculation is provided in the Appendix and it can be written, for example, to approximately halve the computation time by calculating:

e_{w/2}(1:w/2)=e_{w}(1:w/2)+e_{w}(w/2+1:w) (21)

The Fourier power spectrum can be computed/determined (e.g., with the computer arrangement) using MATLAB (e.g., ver. 5.1, 1997, Mathworks) by applying, for example, a Hanning window to the exemplary 8192 discrete point signal. Note that to prevent signal distortion, the traditional Fourier preprocessing method of bandpass filtering, rectification and low pass filtering may not be used (see Background). A fast Fourier transform (FFT) can be then computed from the windowed signal and the power spectrum can be graphed. The t-test and f-test can be used for statistical comparison of means and variances, with significance considered to be, for example, p<0.05 (SigmaPlot ver. 9.0, Systat Software, 2004, and MedCalc ver. 9.5, MedCalc Statistical Software 2008).

### Exemplary Improved Frequency Resolution for Characterization of CFAE

Atrial electrograms were recorded in a series of 20 patients referred to the Columbia University Medical Center cardiac electrophysiology (EP) laboratory for catheter ablation of AF. Ten patients had documented clinical paroxysmal AF, and all 10 had normal sinus rhythm as their baseline rhythm in the EP laboratory. AF was induced by burst atrial pacing from the coronary sinus or right atrial lateral wall, and persisted for at least 10 minutes for those signals included in the retrospective analysis of this study. Ten other patients had longstanding persistent AF, and had been in AF without interruption for 1-3 years prior to the catheter mapping and ablation procedure. The surface electro gram signals were acquired in analog form using the GE CardioLab system (GE Healthcare, Waukesha, Wis.) and filtered from 30-500 Hz with a single-pole band pass filter to remove baseline drift and high frequency noise. The filtered signals were digitally sampled by the system at 0.977 KHz and stored. Although the band pass high end was slightly above the Nyquist frequency, negligible signal energy is expected to reside in this frequency range.

Only signals identified as CFAEs by two cardiac electro physiologists were included in the retrospective analysis. Candidate CFAE recordings of at least 10 seconds in duration were obtained from two sites outside the ostia of each of the four pulmonary veins (PV). Similar recordings were obtained at two sites on the endocardial surface of the left atrial free wall, one in the mid-posterior wall, and another on the anterior ridge at the base of the left atrial appendage. From each of these recordings, 8.4-second sequences (8192 sample points) were analyzed. A total of 240 such sequences were acquired during electrophysiologic analysis—120 from paroxysmal and 120 from longstanding AF patients. Subsequently, only 216 of the recordings were determined to be CFAE, and only these were used for subsequent analysis. As in previous studies, all CFAE signals were normalized to mean zero and unity variance prior to further processing.

### Exemplary Identification of Recurring Patterns in Fractionated Atrial Electrograms

Exemplary electrograms were recorded in a series of twenty patients referred to the Columbia University Medical Center cardiac electrophysiology (EP) laboratory for catheter ablation of AF. Ten patients had documented clinical paroxysmal (acute) AF, with a normal sinus rhythm as their baseline rhythm in the electrophysiology laboratory. Atrial fibrillation was induced by burst pacing from the coronary sinus or the lateral right atrial wall, and the arrhythmia persisted for at least 10 minutes for those signals to be included in the retrospective analysis. Ten other patients had persistent (longstanding) AF, and had been in AF without interruption for 1-6 years prior to the catheter mapping a. Only digitized signals identified as CFAE by two cardiac electrophysiologists were included in the retrospective analysis. The CFAE recordings were obtained from two sites outside the ostia of each of the four PVs. Similar recordings were obtained at two sites on the endocardial surface of the left atrial free wall, one in the mid-posterior wall, and another on the anterior ridge at the base of the left atrial appendage. From each of these recordings, 8.4-second sequences (8192 sample points) were extracted and analyzed. A total of 240 such sequences were acquired—120 from paroxysmal and 120 from longstanding AF patients. Subsequently, only 216 of the recordings were confirmed as CFAE, and only these were used for subsequent analysis. All CFAE signals were normalized to mean zero and unity variance prior to further processing and ablation procedure. Bipolar electrograms of at least 10 seconds in duration, recorded from the distal ablation electrode during arrhythmia, were bandpass filtered by the system at acquisition to remove baseline drift and high frequency noise (30-500 Hz), sampled at 977 Hz, and stored. Although the bandpass high corner was slightly greater than the Nyquist frequency, negligible signal energy resides in the region.

Only digitized signals identified as CFAE by two cardiac electrophysiologists were included in the retrospective analysis. The CFAE recordings were obtained from two sites outside the ostia of each of the four PVs. Similar recordings were obtained at two sites on the endocardial surface of the left atrial free wall, one in the mid-posterior wall, and another on the anterior ridge at the base of the left atrial appendage. From each of these recordings, 8.4-second sequences (8192 sample points) were extracted and analyzed. A total of 240 such sequences were acquired—120 from paroxysmal and 120 from longstanding AF patients. Subsequently, only 216 of the recordings were confirmed as CFAE, and only these were used for subsequent analysis. All CFAE signals were normalized to mean zero and unity variance prior to further processing.

### Exemplary Spectral Profiles of Complex Fractionated Atrial Electrograms

Atrial electrograms were recorded in a series of 20 patients referred to the Columbia University Medical Center cardiac electrophysiology (EP) laboratory for catheter ablation of AF. Ten patients had documented clinical paroxysmal AF, and all 10 had normal sinus rhythm as their baseline cardiac rhythm in the cardiac electrophysiology laboratory. AF was induced acutely by burst atrial pacing from the coronary sinus or right atrial lateral wall, and allowed to persist for at least 10 minutes prior to data collection. Patients in whom only short runs of AF were inducible were excluded from this study. Ten other patients had longstanding persistent AF, and had been in AF without interruption for 6 months to 6 years prior to their catheter mapping and ablation procedure.

The duration of uninterrupted AF in these patients was estimated as the period from the time of recurrence of AF after the last DC cardioversion (which converted AF to sinus rhythm) to the day of the catheter ablation procedure. Bipolar atrial mapping was performed with a NaviStar ThermoCool catheter, 7.5 F, 3.5 mm tip, with 2 mm spacing between bipoles (Biosense-Webster Inc, Diamond Bar, Calif., USA). The electrogram signals were acquired using the GE CardioLabsystem (GE Healthcare, Waukesha, Wis., USA), and filtered at acquisition from 30 to 500 Hz with a single-pole bandpass filter to remove baseline drift and high frequency noise. The filtered signals were digitally sampled by the system at 0.977 KHz and stored. Although the bandpass high end was slightly above the Nyquist frequency, negligible CFAE signal energy resides in this frequency range 10. Only signals identified as CFAEs by 2 cardiac electrophysiologists were included in this analysis. 9, 10, 12 CFAE recordings of at least 10 seconds in duration were obtained from 2 sites outside the ostia of each of the 4 PVs. Similar recordings were obtained at 2 LA free wall (FW) sites, one in the mid posterior wall (POS), and another on the anterior ridge at the base of the LA appendage (ANT). The mapping catheter was navigated in these prespecified areas until a CFAE site was identified. In 1 patient with clinical paroxysmal AF, during acutely induced AF, no recording site outside the PVs with recordings satisfying CFAE criteria for at least 10 seconds could be detected. Therefore, data from this patient were not included in the following analysis. From each of the exemplary recordings described above, when a CFAE sequence over about 16.8 s was recorded during AF, 2 consecutive 8.4 s series were extracted and analyzed. Only sites at which the CFAE criteria were maintained during the recorded sequence were used for analysis. A total of 204 sequences 90 from paroxysmal and 114 from longstanding AF patients, all meeting the criteria for CFAE—were chosen for this study and included in the following analysis. As in the previous studies, to standardize the morphological characteristics, all CFAE signals were normalized to mean zero and unity variance (average level=0 volts, standard deviation=1).

To remove the second harmonic, which is usually the predominant sub- or superharmonic, an exemplary antisymmetry technique was applied to each ensemble average.

### Exemplary Results

### Exemplary Orthogonality of the Ensemble Basis

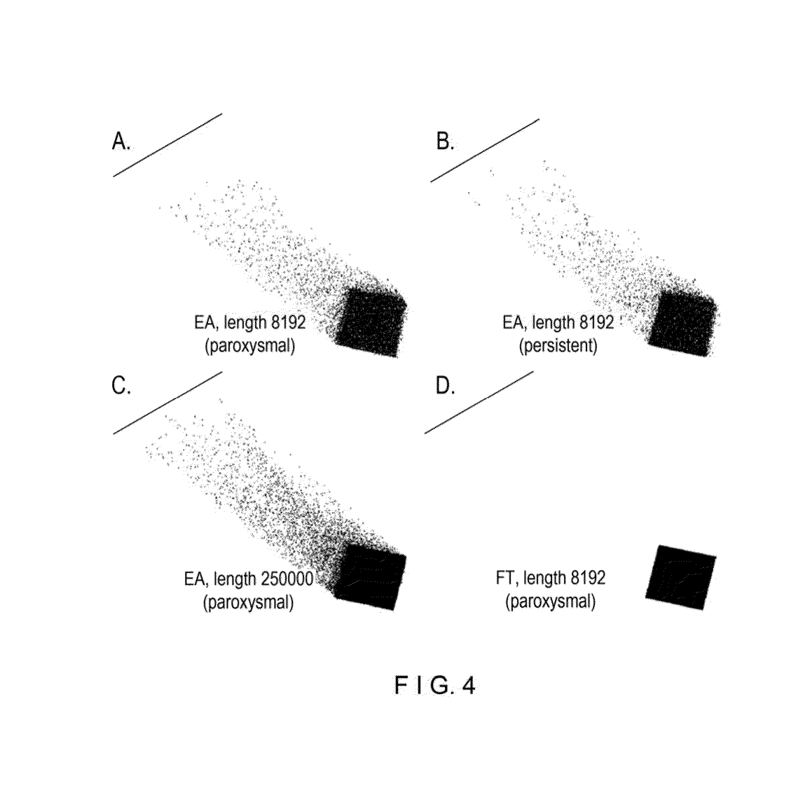

An exemplary result of the inner product measurement (exemplary Eq. (19)) can be shown, for example, in FIG. 4, which can be generated using, e.g., map3d, an interactive scientific visualization tool for bioengineering data devised by the Scientific Computing and Imaging Institute, University of Utah. (See, e.g., Reference [24]). In each panel the DP magnitude scale can increase, for example, from 0 to 1 from lower right to upper left. In panels A-C the exemplary result for the exemplary ensemble method can be shown, computed for all bases a500-a20 (e.g., 481 basis vectors ranging from 2 Hz-50 Hz). In panel A (e.g., paroxysmal AF) DP values, for example, are near zero when i≠j, (e.g., fuzzy square region). A line can be formed at unity magnitude at upper left, corresponding to i=j (e.g., autocorrelation). Where i and j can be harmonically related, the DP magnitude can be intermediate (e.g., few scattered points between lower right and upper left).

A similar result can be obtained for the persistent AF signal (B). For all values i≠j including those that were harmonically related, the mean normalized inner product can be, for example, 0.0075±0.0510 for 108 paroxysmal CFAE and 0.0077±0.0509 for 108 persistent CFAE signals (e.g., <1% of the magnitude when i=j). For N=8192, random cancellation of uncorrelated components may have been incomplete. As a further test, the basis vectors for the paroxysmal CFAE signal can be extended, for example, to N=250,000 in length, and the resulting inner products can be graphed in panel C. In this panel, when i≠j and no harmonic relationship exits, dp=0.0 (e.g., square region is solid rather than fuzzy i.e. there can be complete cancellation of random components). Thus, the exemplary ensemble basis can be orthogonal except for small integer harmonic relationships. For comparison, the dp using Fourier bases (N=8192) can be shown in panel D. Since the sinusoidal basis can be antisymmetric about the x-axis, the inner product can be zero when i≠j, even for harmonic relationships.

Exemplary Spectral Analysis of Synthetic Drivers with Phase Noise and Interference

FIGS. 5A and 5B illustrate, for example, Fourier and ensemble spectra of the three synthetic drivers when interference is added (p=0x, i=5×). Most of the spectral components are typically caused by the drivers, with the interference contributing to the noise floor (e.g., compare FIGS. 5B and 3D from 2-10 Hz). The location of synthetic driver peaks are noted in FIGS. 5A and 5B by asterisks. Portions of the noise floor can extend beyond two driver peaks in the Fourier spectrum (see FIG. 5A). The driver peaks can be all higher than the noise floor in the ensemble spectrum (see FIG. 5B). There can be more detail at the lower end of the ensemble spectrum due to the w=1/f relationship (exemplary Eq. (6)), and subharmonics can also be evident. The exemplary result for measurements with the various additive noise combinations and interferences is shown, for example, in Table 1.

TABLE 1
Sum of Ranks of 3 Driver Frequencies

| p | 1 | Fourier | Ensemble | MN | SD |
|---|---|---|---|---|---|
| 1× | 1× | 7.12 ± 1.41 | 6.71 ± 1.08 | .005 | NS |
| .5× | 2× | 7.03 ± 0.48 | 6.31 ± 0.10 | <.001 | <.001 |
| .3× | 3× | 7.88 ± 0.30 | 6.73 ± 0.08 | <.001 | <.001 |
| 0× | ±10× | 10.24 ± 3.37 | 8.82 ± 3.08 | <.001 | NS |

p = gain of added phase noise i = gain of added interference.
MN, SD = significance of mean and standard deviation.

In the first and second columns of Table 1, the phase and interference multipliers, respectively, are shown. In the third and fourth columns, mean±standard deviation in the sum of ranks for D1, D2, and D3 are shown. The significance of the differences are noted in the last two columns. All of the means can be significantly different, with the synthetic drivers, for example, being more highly ranked in the ensemble spectra (total rank is closer to 6). The standard deviation in total rank, e.g., the variability, can be higher in Fourier as compared with ensemble, with a significant difference in two cases.

### Exemplary Identification of Synthetic Driver Morphology

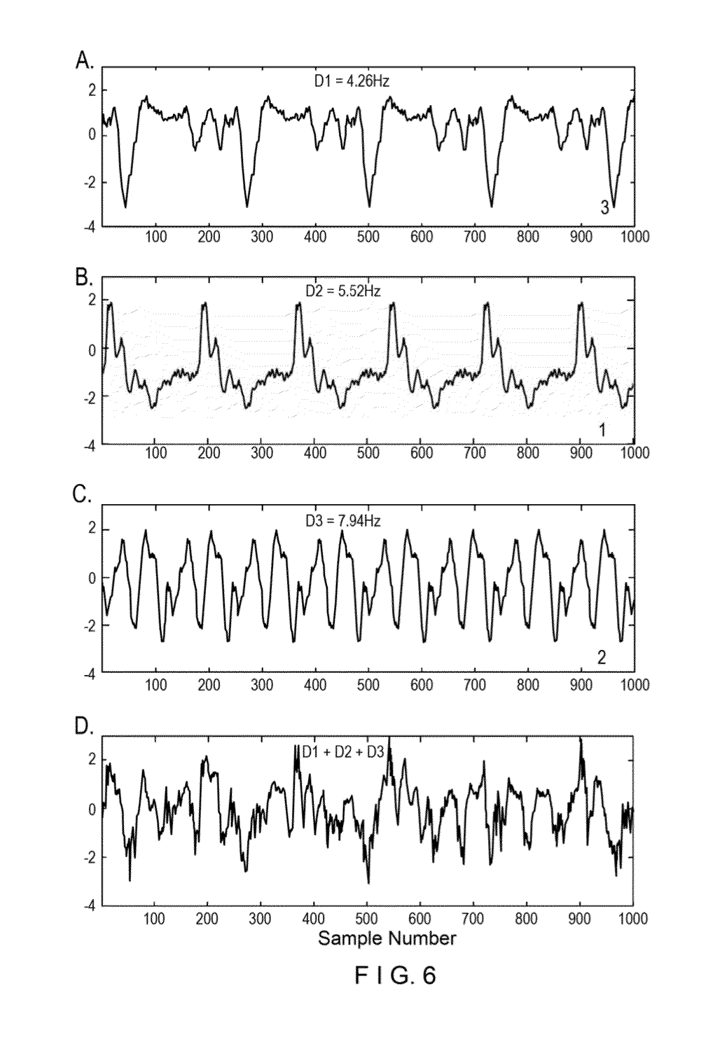

FIGS. 6A-6D show graphs for an example of the top three basis vectors (e.g., panels A-C) constructed from synthetic drivers with noise and interference added (e.g., weighting p=0.3×, i=3×, panel D). The basis vectors in FIGS. 6A-6C can be reflective of the corresponding original drivers depicted in FIG. 2. Some smoothing can occur in the fine detail due to the phase noise (jitter) that can be added to the drivers. The 4.37 Hz, 5.65 Hz, and 8.13 Hz bases can be ranked the 3rd, 1st, and 2nd highest peaks, respectively, in the ensemble power spectrum, as is noted at bottom right in each panel A-C. For the noise set (p=0×, i=5×) the corresponding basis vectors can estimate the FIG. 2 drivers, as there was no added jitter (not shown). For the exemplary 216 tests with phase noise and interference (p=0.3×, i=3×) the average mean squared error can be 0.091±0.020 mV²/ms, while for additive interference only (p=0×, =5×) it can be only 0.0049±0.0042 mV2/ms. These errors can be, for example, <10% of the power in the normalized drivers (1.0 mV2/ms). Thus, in the presence of jitter and/or interference, the morphology of independent drivers in CFAE can be extractable using the ensemble basis.

Exemplary Degradation DF in CFAE with Additive Random Noise

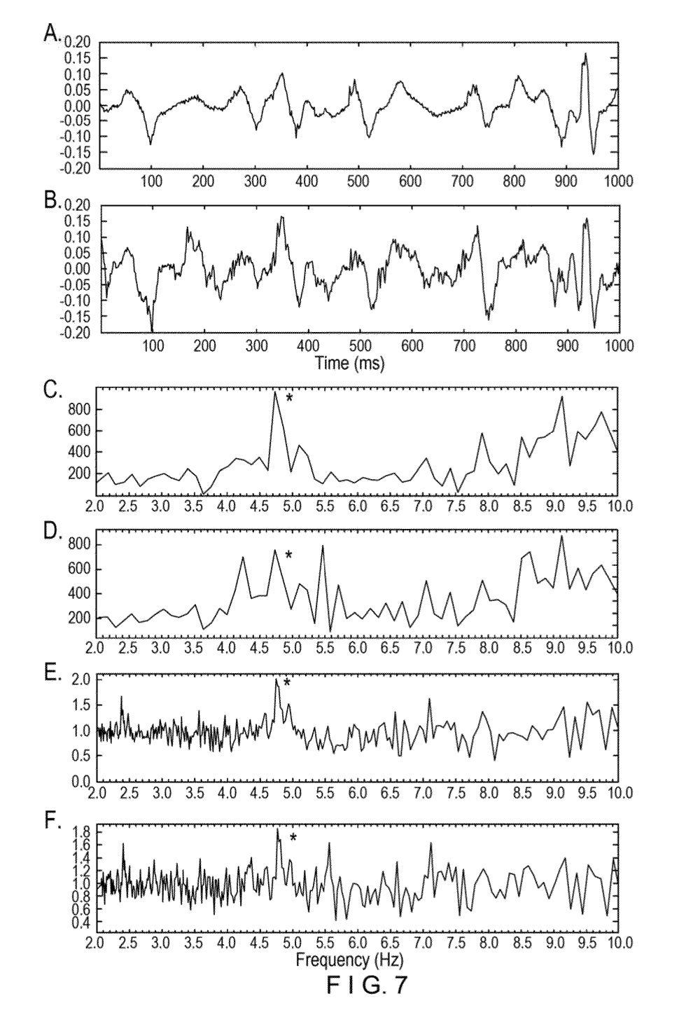

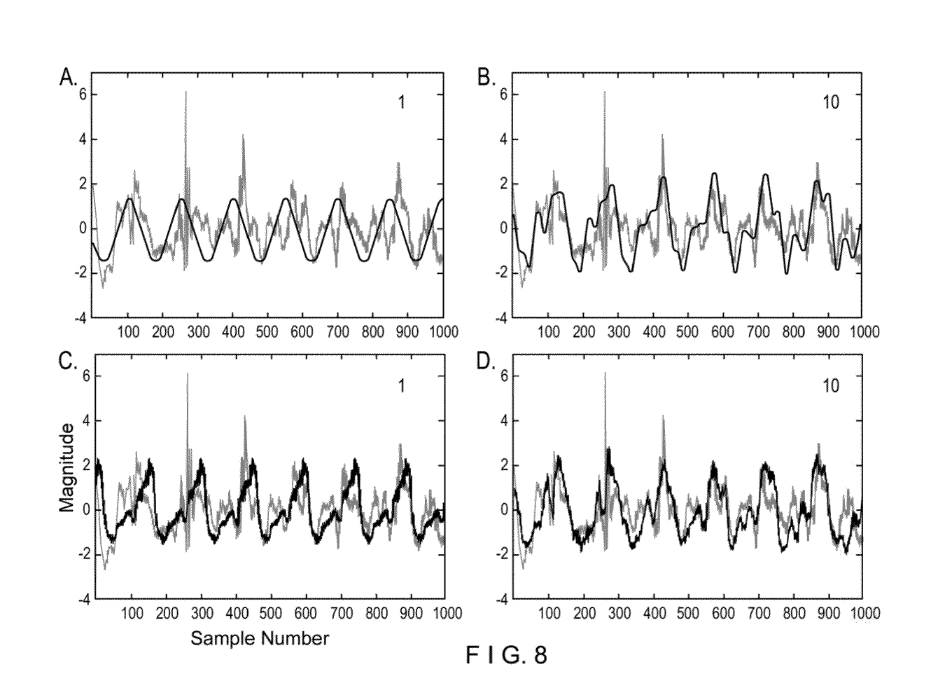

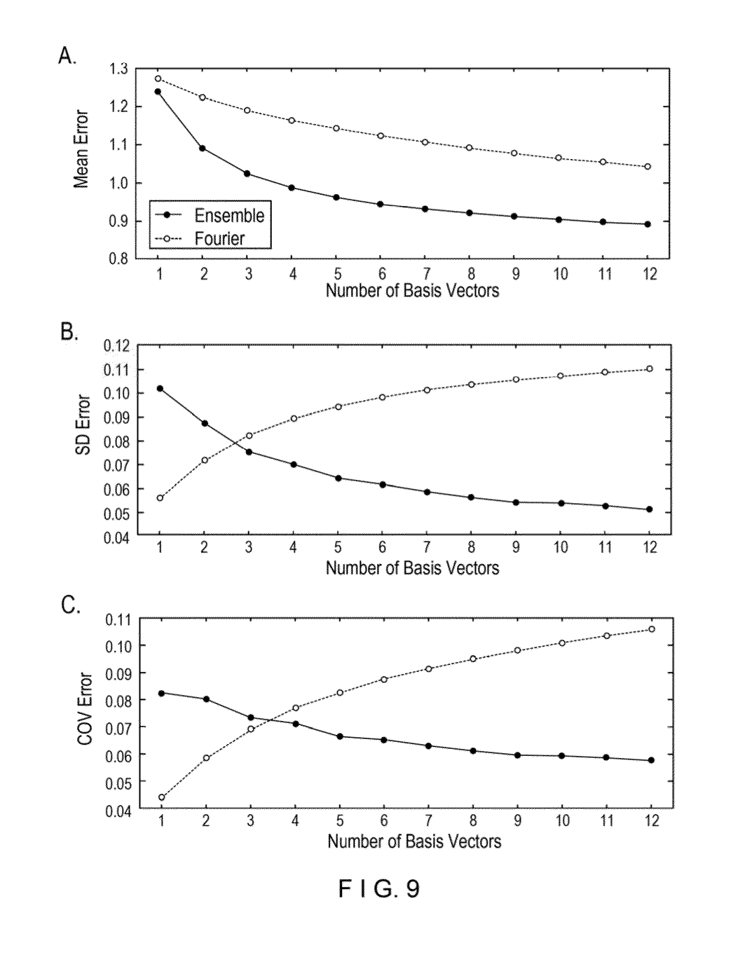

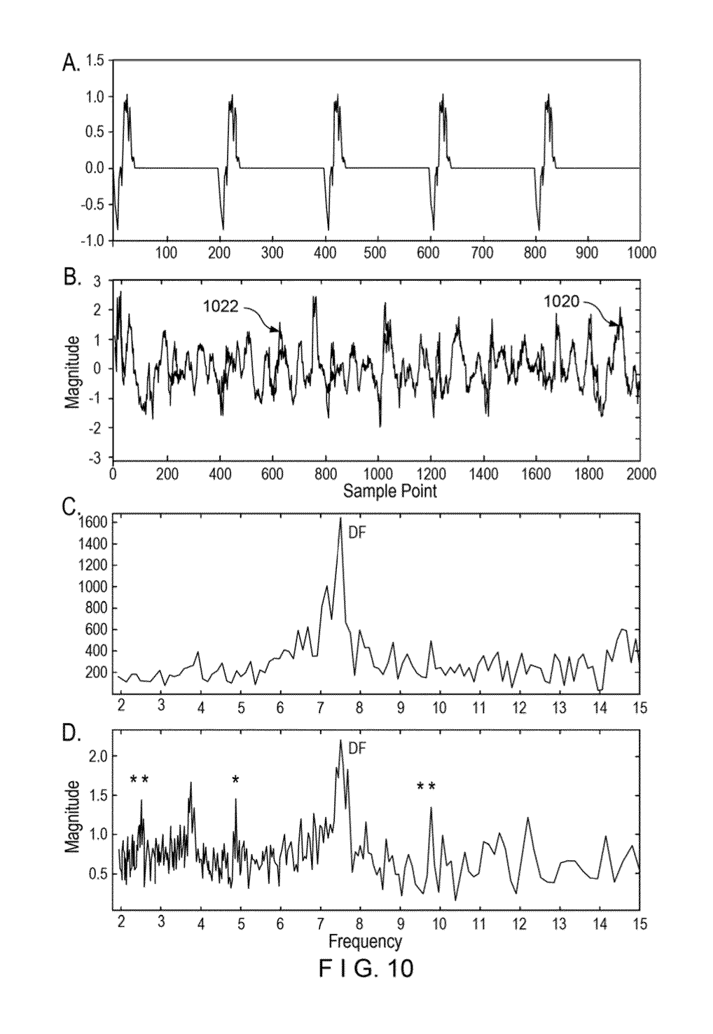

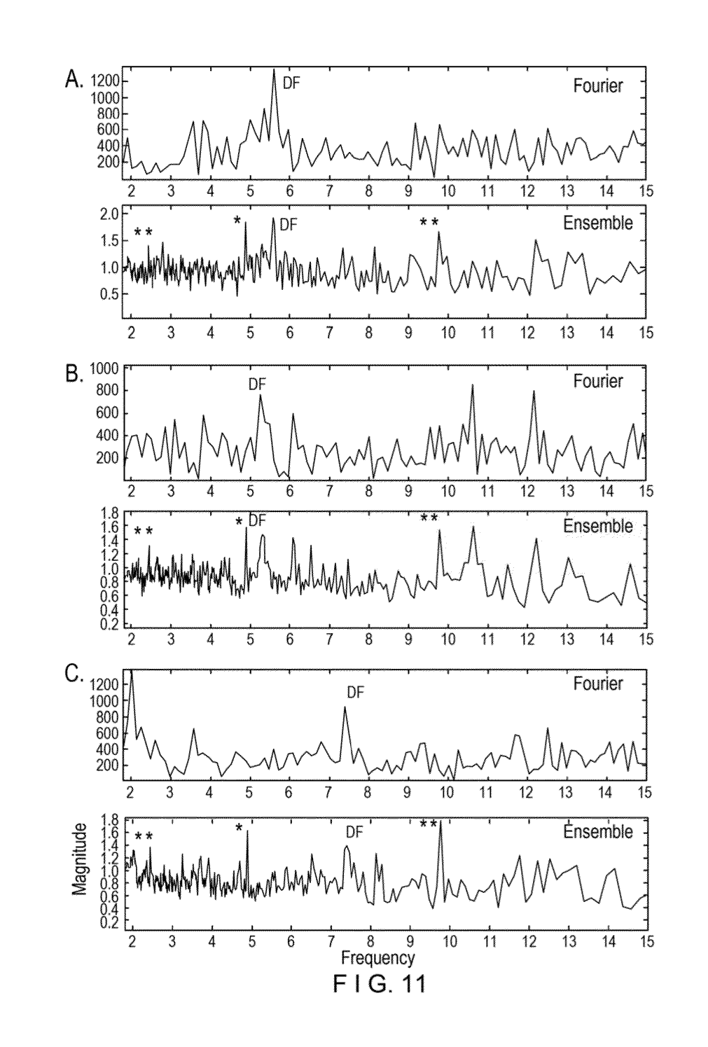

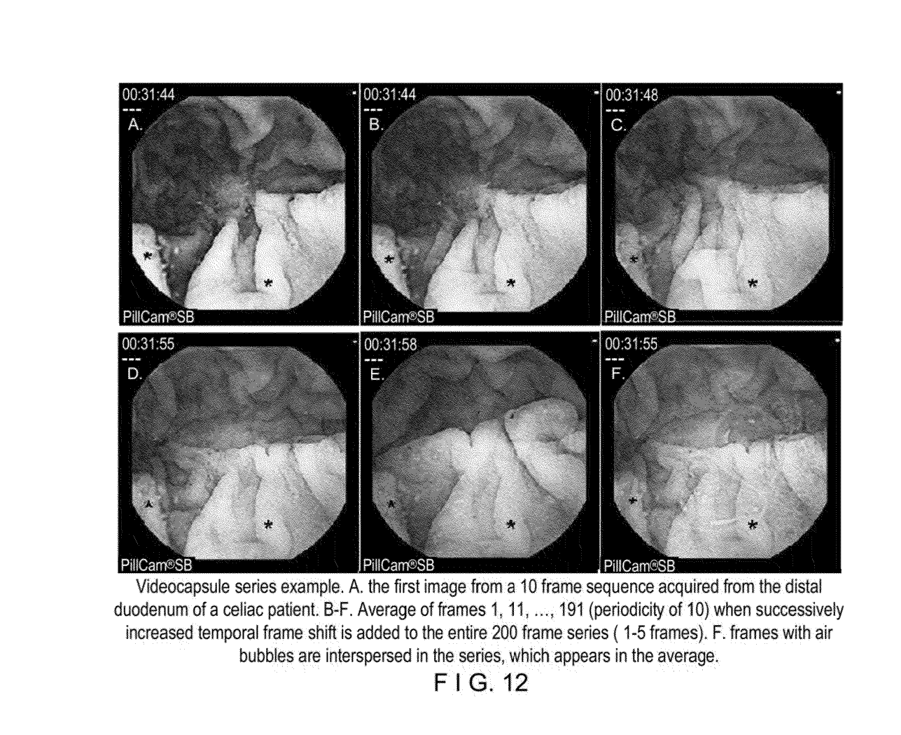

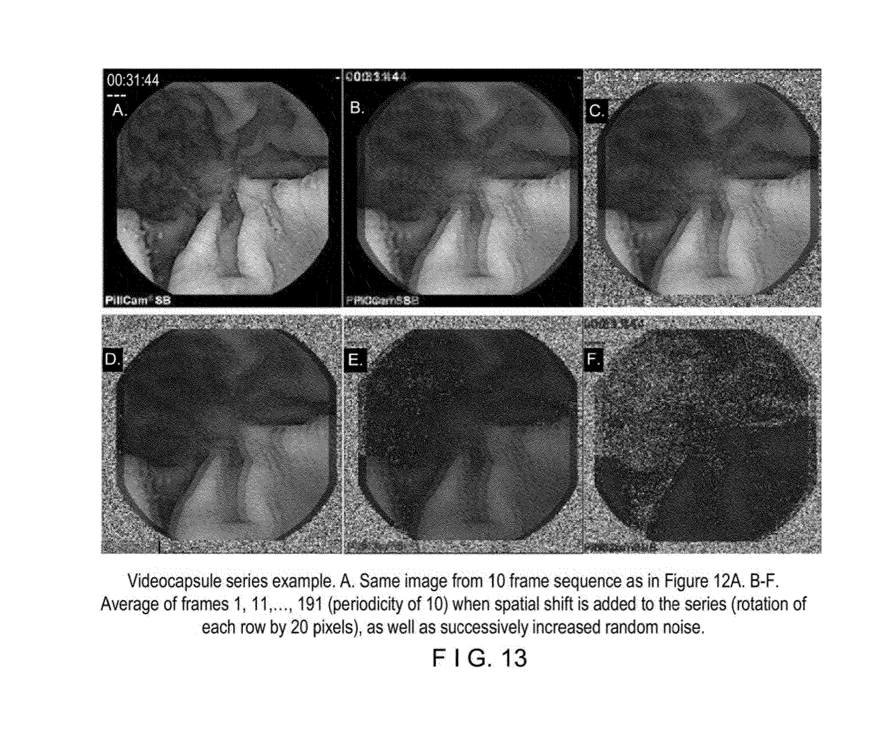

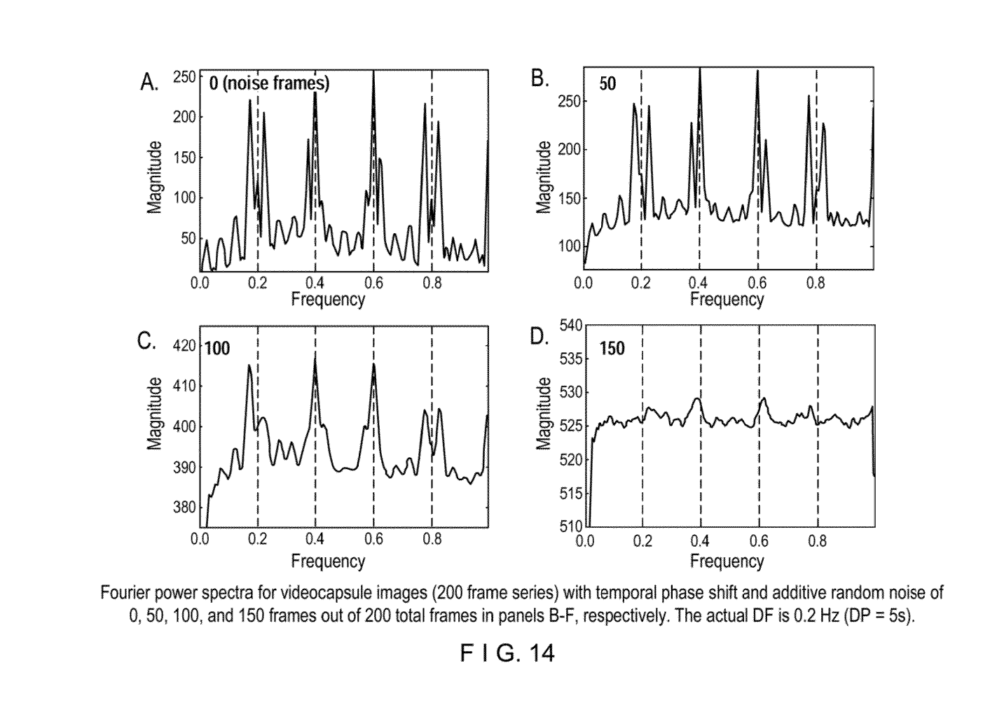

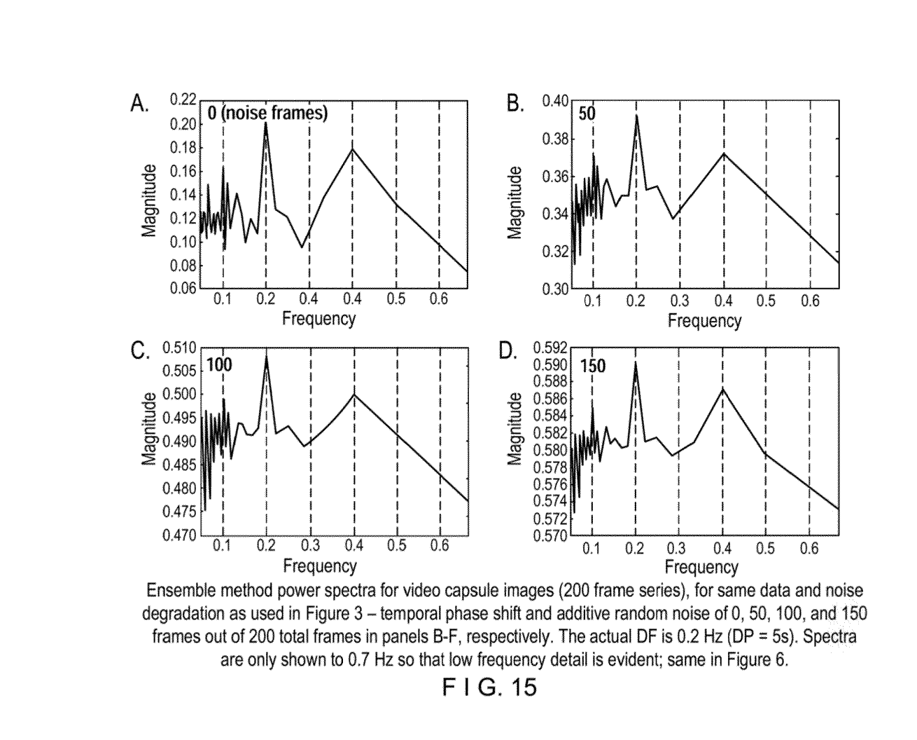

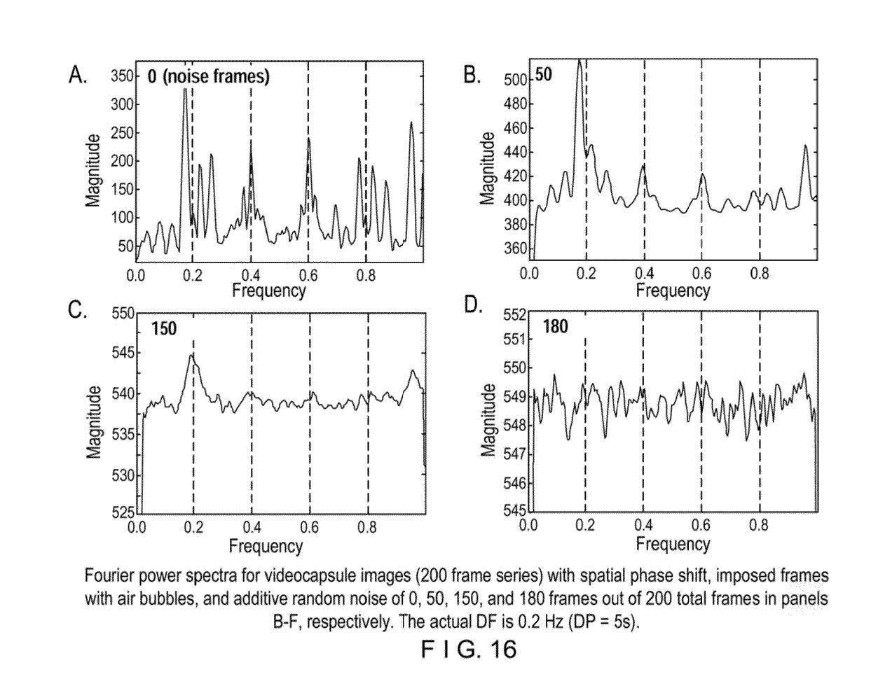

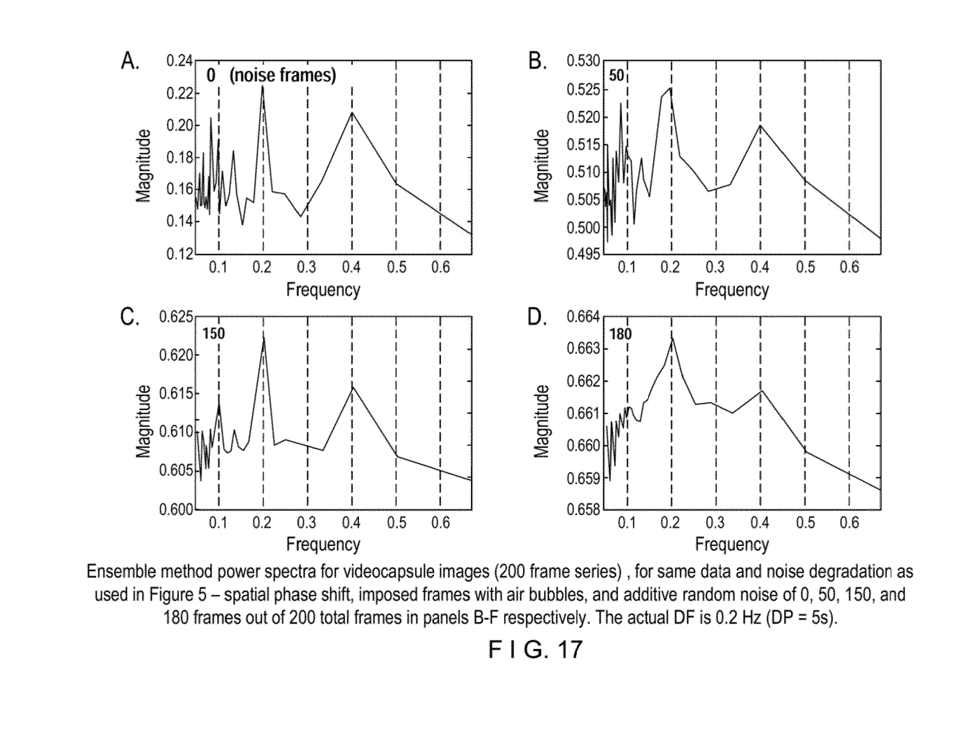

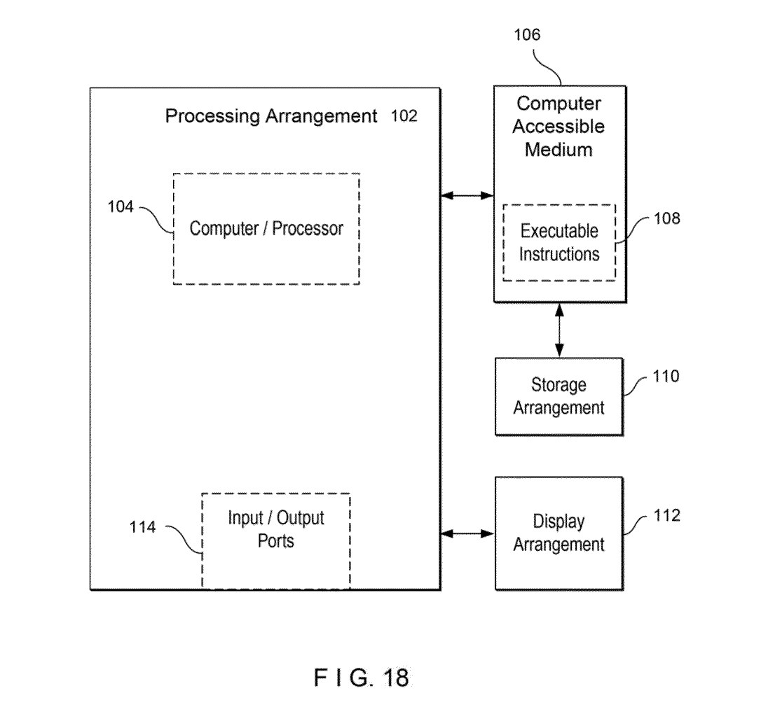

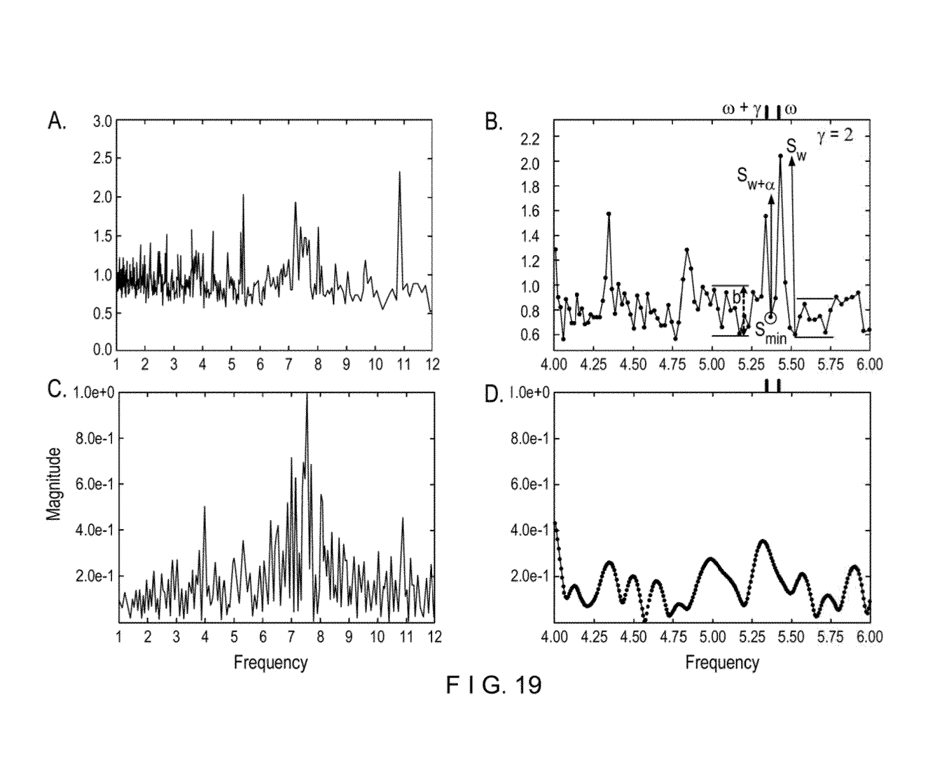

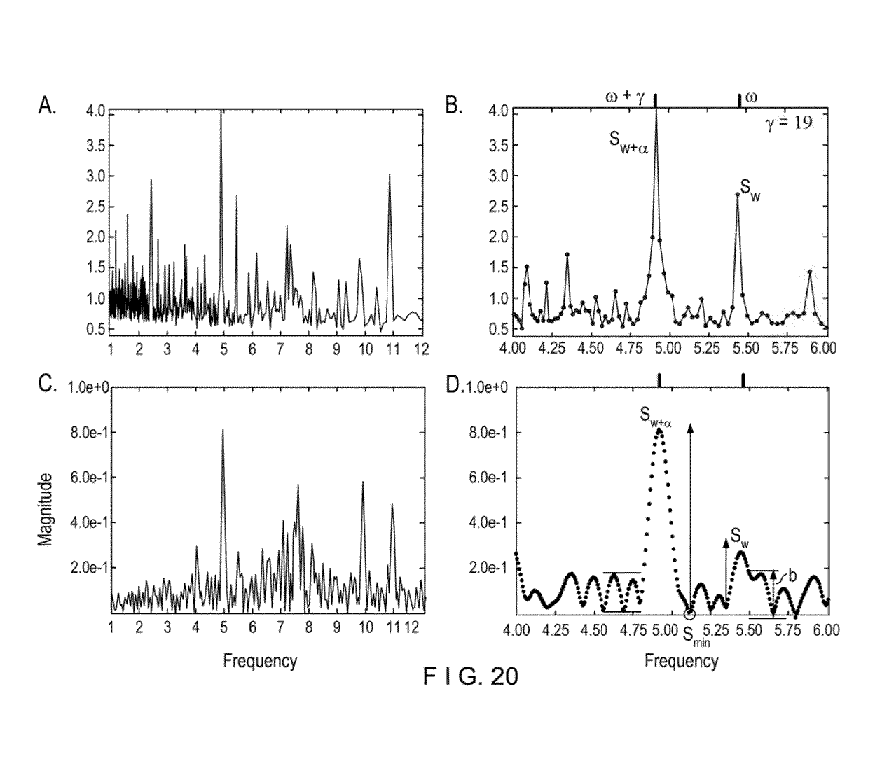

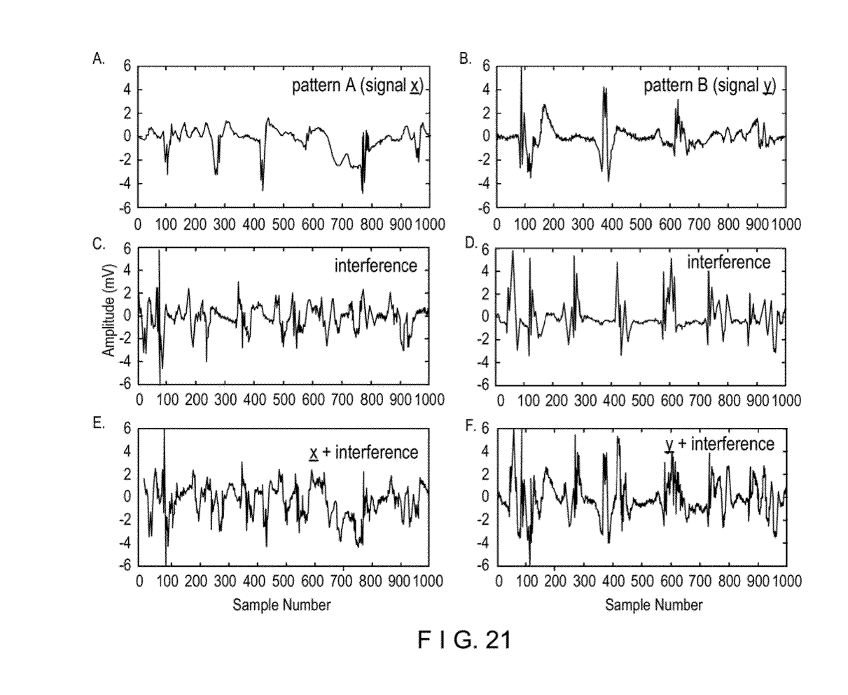

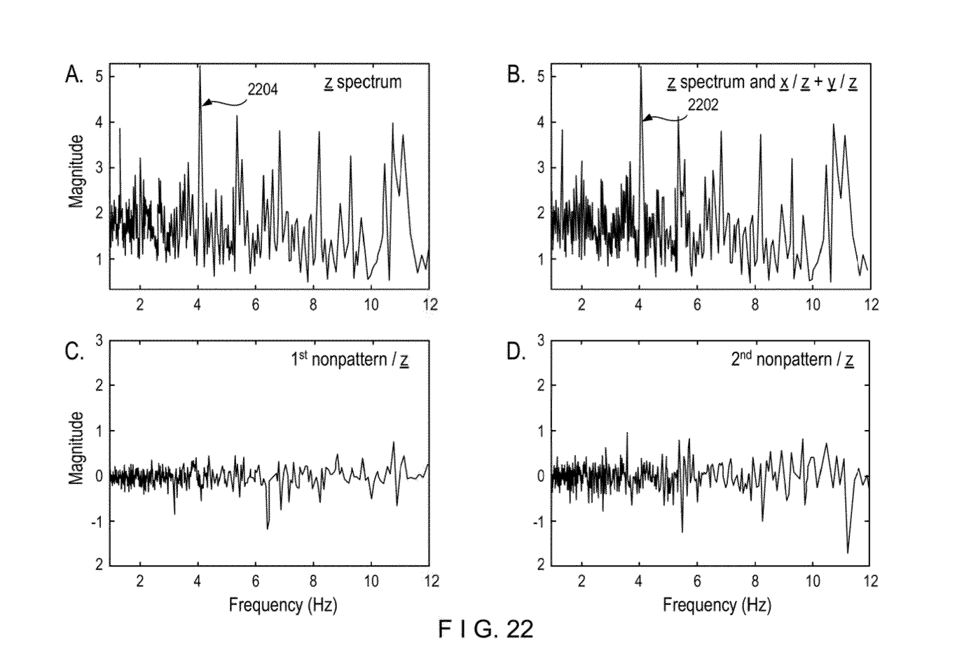

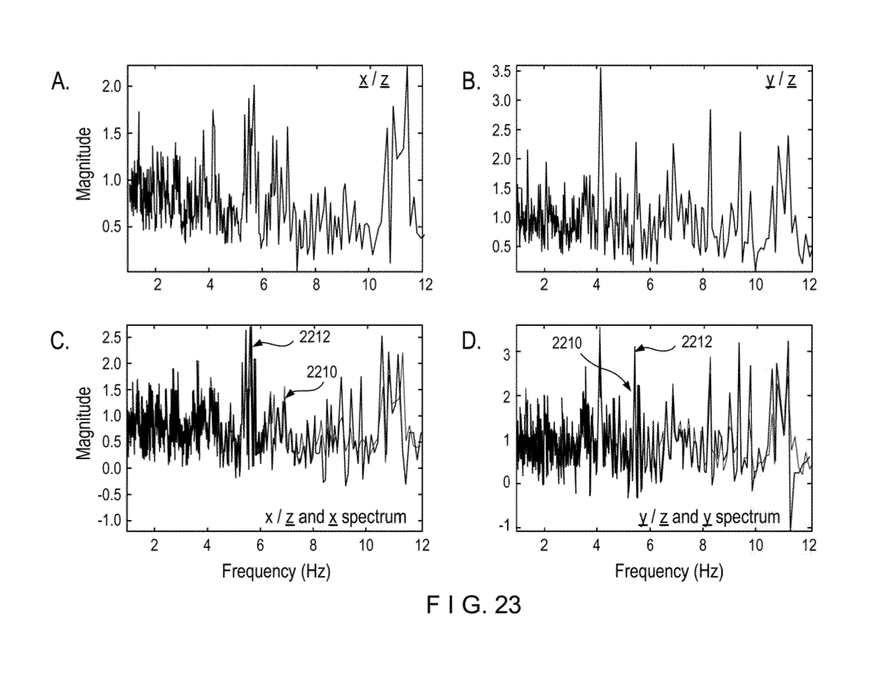

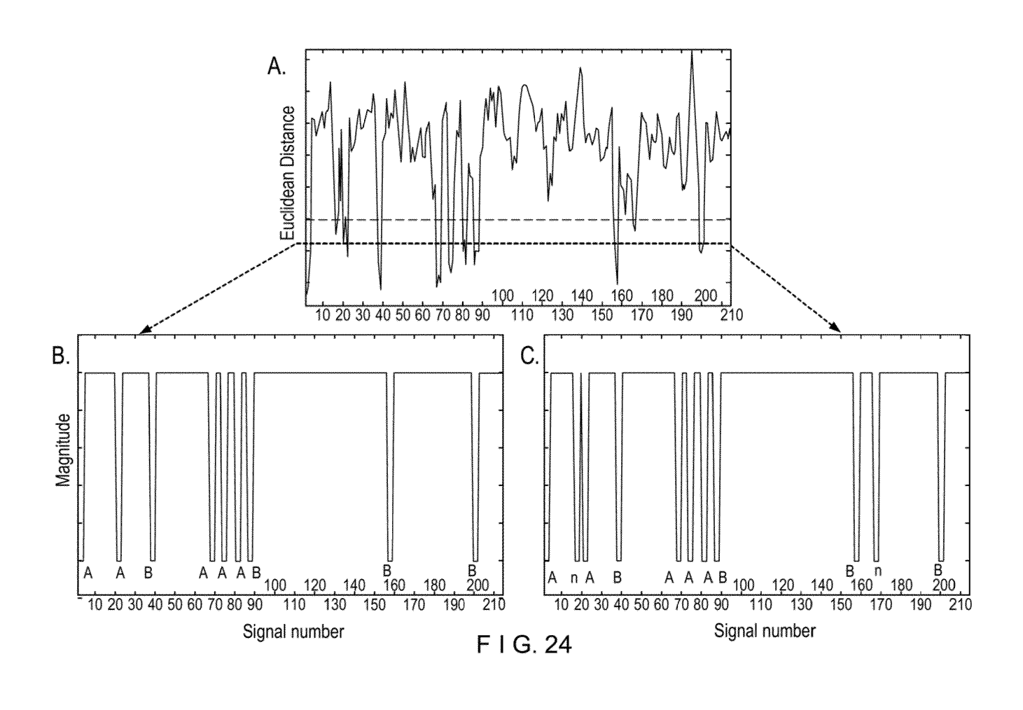

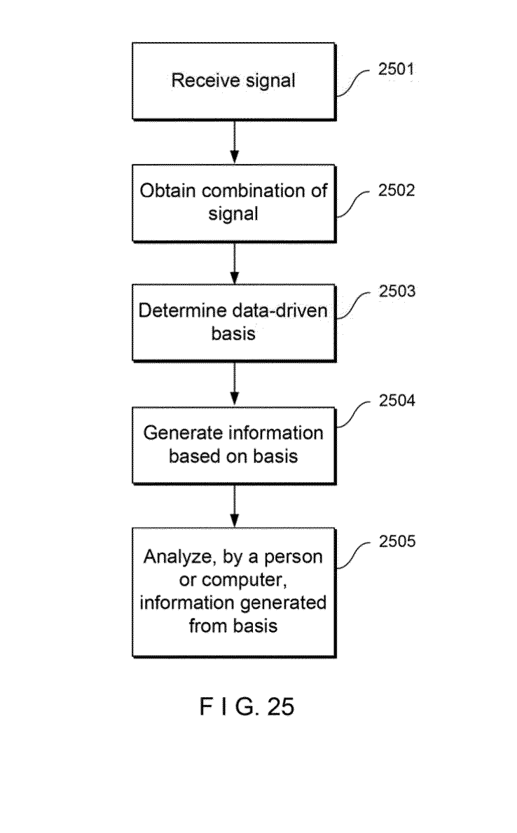

For random noise added with SD=±16 ms, the mean absolute difference in DF before versus after addition of a random noise vector can be, for example, 0.35+0.02 Hz for Fourier spectral analysis versus 0.09±0.05 Hz for ensemble spectral analysis (p<0.001). For random noise added with SD=±32 ms, the mean absolute difference in DF before versus after addition of a random noise vector can be, for example, 0.68+0.10 Hz for Fourier spectral analysis versus 0.53±0.13 Hz for ensemble spectral analysis (e.g., p=0.01). An example is shown in FIGS. 7A-7F for a CFAE signal from the anterior left atrial free wall of a paroxysmal AF patient. Panels of FIGS. 7A and 7B show, for example, the CFAE prior to and after addition of random noise with SD=±0.16 mV, while panels of FIGS. 7C and 7D and of FIGS. 7E and 7F show the corresponding Fourier and ensemble averaging spectra. In each spectrum the DF is noted by an asterisk. After noise addition, the DF peak can be the third highest in the Fourier spectrum (panel D) but it can remain the highest peak in the ensemble averaging spectrum (panel F). Thus, as shown in FIGS. 7A-7F and Table 1, the DF peak in ensemble spectral analysis can be less affected by and more robust to random white additive noise might occur due to motion artifact, electrical component oscillation, and/or broken wire leads.

### Exemplary CFAE Reconstruction

An example of the Fourier basis vectors a_{w} constructed from e_{w} with 1st and 10th highest power is shown, for example, in the graphs of FIGS. 8A and 8B from a paroxysmal CFAE signal acquired from the LIPV. The corresponding exemplary ensemble basis vectors for this same signal are shown, for example, in FIGS. 8C and 8D. As the Fourier basis is typically general and sinusoidal, the estimates can approximate the signal with relatively large error (FIGS. 8A and 8B). However the exemplary ensemble basis can be data-generated and constructed from the first moment of the signal, so that it can be more estimative of the actual signal even when, for example, only the single most important basis vector is used (see FIG. 5C). There can be substantial overlap with the actual CFAE trace when 10 basis vectors can be used for reconstruction (e.g., FIG. 8D). For the 12 reconstruction vectors, the root MSE averaged 1.13±0.07 mV for Fourier versus 0.98±0.10 mV for ensemble (p<0.001). The reconstruction error can be also lower for ensemble versus Fourier for each individual reconstruction using 1-12 bases (p<0.002).

The statistical relationships are illustrated, for example, in FIG. 9. The mean error in reconstruction for ensemble averaging can decrease more rapidly as compared with Fourier (see the graph of FIG. 9A). The standard deviation in the reconstruction error for the CFAE is shown, for example, in the graph of FIG. 9B. The standard deviation can fall off rapidly for ensemble averaging and can increase rapidly for Fourier. At ≧3 basis vectors, the standard deviation in reconstruction error can be lowest for ensemble averaging. This means that the ability of ensemble averaging to consistently reconstruct CFAEs (panel of FIG. 9B) with a similarly minimal level of error (panel of FIG. 9A) can be mostly improved as compared with Fourier reconstruction. Similarly, the coefficient of variation, which can be the standard deviation divided by the mean (Figure of FIG. 9C), can fall off for ensemble average reconstruction but it actually increases for Fourier reconstruction.

### Exemplary Single Driver Test

The 5 Hz transient described above is shown, for example, in FIG. 10A and its addition to a CFAE is shown, for example, in FIG. 10B, trace 1022. For comparison, the original CFAE is shown as trace 1020 in panel B and it can be the same trace as in FIG. 1A. The Fourier and ensemble average power spectra are shown, for example, in FIGS. 10C and 10D, respectively. Although both spectra show a DF at ˜7.5 Hz and a smaller peak at ˜3.9 Hz (which may be generated by an independent driver), only the ensemble average power spectrum, for example, indicates presence of the artificial transient at 5 Hz (noted by *; with super- and subharmonics noted by **). For all CFAEs, the 5 Hz transient was identified, for example, in 216/216 ensemble averaging spectra (100%) but was only present, for example, in 82/216 Fourier spectra (38.0%). Additional examples are provided, for example, in the graphs of FIGS. 11A-11C. In each pair of Fourier and ensemble spectra, both have the same DF, for example, in the range 3-10 Hz. However, the 5 Hz transient can be evident in the ensemble averaging spectra (again noted by *; with super- and subharmonics noted by **). Thus, ensemble averaging but not Fourier spectral analysis can be sensitive to the presence of far-field and/or low-power drivers which affect CFAE over short intervals.

### Exemplary Improved Frequency Resolution for Characterization of CFAE

A graph of an exemplary power spectrum using the new spectral estimation technique is shown in FIG. 19A. Note that the highest frequency resolution occurs at lower frequencies due to the 1/w relationship of resolution to frequency for this method. By comparison, the Fourier power spectrum is uniform in resolution across the range (FIG. 19C). FIGS. 19B and D show close-ups of the respective spectra in the range of the synthesized components. The actual synthesized components have frequencies of 5.34 Hz (ω+γ=183 sample points at 977 Hz sampling rate) and 5.43 Hz (ω=180 sample points), noted by vertical bars at the tops of panels 3B and 3D. The two components are correctly resolved by the new technique (panel 3B), that is, w=ω and w+α+γ. However, Fourier analysis does not resolve at this component spacing (panel 3D). In FIG. 20, using the same CFAE and with the high frequency remaining at 5.43 Hz (ω=180 sample points), the exemplary graphical result is shown for γ=19 when the low frequency is 4.91 Hz (ω+γ=199 sample points). The spectrum and close-up using an exemplary embodiment of the method according to the present disclosure are shown in FIG. 20A-B, and the frequency components are readily resolved as shown in an exemplary graph of FIG. 19A-B. The Fourier spectrum is shown in FIG. 20 C-D and now distinct peaks appear (see FIG. 20D), meeting the exemplary criteria set forth in the Methods. This was the exemplary minimum distance γ at which two corresponding Fourier spectral peaks met the criteria, and therefore the resolution for the Fourier spectrum. The measurements for S_{w+α}, S_{w}, Sₘᵢₙₙ, and b are shown.

### Identification of Recurring Patterns in Fractionated Atrial Electrograms

FIGS. 21A-21D illustrate exemplary graphs of signals and additive exemplary interferences. Identical scales can be used in all panels. For example, in panel A is shown a CFAE from the right superior pulmonary vein ostia in a paroxysmal AF patient. In FIG. 21B, an exemplary CFAE graph is illustrated from the anterior left atrial free wall in another paroxysmal AF patient. Both signals have mostly continuous activation, and the large deflections have different shape and timing at each occurrence. Only 1000 of 8192 sample points are shown for clarity (approximately 1 second), although 8192 points were used for the calculations described in the Methods. The signals illustrated in the exemplary graphs of FIGS. 21A and 21B can be used as patterns, which can be made to occur, e.g., five and four times, respectively, in the final data set of, e.g., 214 signals used for analysis. Examples of additive interference are shown in corresponding the exemplary graphs shown in FIGS. 21C and 21D. The interferences are each a combination of two AF signals unrelated to signals shown in FIGS. 21A and 21B. The same or similar exemplary patterns after addition of the interferences are shown in the corresponding exemplary graphs shown in FIGS. 21E and 21F. With the additive interferences, the original signals are almost completely unrecognizable visually. Most of the original signal deflections are masked by interference.

The exemplary spectrum of the combined exemplary patterns illustrated in FIGS. 21A and 21B is shown in FIG. 22A in the range 1-12 Hz, where pattern A (signal x)+pattern B (signal y) form the combined signal z. For example, several prominent peaks are shown in the exemplary spectrum of z, likely related to individual components of the two signals. The transform coefficients of x and y with respect to the basis vectors of z were separately calculated and then added together and plotted as a trace 2202 in FIG. 22B, shown with exemplary overlapping z spectrum 2204 shown in FIG. 22A. There is perfect overlap in accord with. In contrast, when the spectral signatures of two other signals not related to x or y are obtained with respect to z, their magnitude throughout the frequency range is relatively small and the transform coefficients are both positive and negative (see exemplary FIGS. 22C and 22D; same or similar 5-unit range in ordinate scale as shown in FIGS. 22A and 22B).

To further elucidate the exemplary process, when the spectral signatures of x and y with respect to z are separately plotted (see FIGS. 23A and 23B, respectively), there are similarities to the z spectrum shown in FIG. 22A. Therefore, exemplary elements of the z spectrum (FIG. 22A) are maintained in the spectral signatures of x and y (see FIGS. 23A and 23B, respectively), suggesting that the Euclidean distances between them will be relatively small. In contrast, the elements of the z spectrum are not maintained in the spectral signatures of random interferences, such as those shown in FIGS. 22C and 22D, suggesting that the Euclidean distances between them will be relatively large. Finally, the spectral signatures of x and of y with respect to z, shown again as traces 2210 in FIGS. 23C and 23D, are similar, but not the same, as the spectra of x and y, which are denoted as traces 2212 shown in FIGS. 23C and 23D. Based on the exemplary graphs shown in FIGS. 22A-22D and 23A-23D, the spectral signatures of x and y with respect to z are related to the actual frequency content in signals x and y. However, the x and y spectra do not resemble each other since they are uncorrelated.

The Euclidean distance between the spectral signatures of each of 214 signals with differing additive interference, versus the spectrum of the mean signal containing two patterns A and B, is shown in an exemplary graph of FIG. 24A. A number of downward projections are illustrated in FIG. 24A, which indicate increased correlation and possible instances of pattern recurrence. If the lower threshold is used, nine possible instances of repetitive patterns are selected (shown in binary form in FIG. 24B). When the upper threshold is used, eleven possible instances of repetitive patterns are selected (shown in binary form in panel C). The detected pattern type (A or B) or non-pattern (n) are shown at the bottom of FIGS. 24B and 24C. The selection of a threshold higher along the ordinate axis in the Euclidean distance graph shown in FIG. 24A can facilitate the detection of more candidate patterns. However, whatever threshold is used, to determine and identify the presence of actual recurring patterns necessitates the last step at lower right in the pattern recognition flow diagram of FIG. 1, i.e., the exemplary spectral signatures of the signals selected by threshold shown in FIGS. 24A-24C can be compared. Due to the exemplary constructing of the signals with the exemplary interference, as described herein, each downward projection shown in FIGS. 24B and 24C can represent a set of three exemplary successive signals with pattern, of which the middle was used for an exemplary statistical calculation.

The Euclidean distances for the exemplary pairings of spectral signatures using the upper exemplary threshold shown in FIG. 24A (shown in binary form in FIG. 24C) are provided in Table 2. The first column and first row in Table 2 indicate the actual pattern that was selected by the upper threshold in FIGS. 24A-24C, and correspond to the sequence shown in FIG. 24C. Since the two patterns A and B occurred only nine times in the sequence, two of the selections shown in the exemplary graphs of FIGS. 24A-24C, top threshold, were of non-patterns (n). In the case of the pairing of a spectral signature from a particular signal with itself, the Euclidean distance can be zero (main diagonal in Table 2). There is an exemplary symmetry above and below the main diagonal (half the table is redundant). Smaller values in Table 2 can indicate shorter Euclidean distances, i.e., spectral signatures that are more similar. The Euclidean distances tend to be small for spectral signatures of pattern A embedded in one interference versus pattern A embedded in another interference, and similarly for pattern B embedded in one interference versus pattern B embedded in another interference. The Euclidean distances tend to be large for spectral signatures of pattern A versus pattern B embedded in interference, for spectral signatures of patterns A and/or B embedded in interference versus non-patterns (e.g., interference only), and for spectral signatures of non-pattern versus non-pattern. Thus, the exemplary patterns and non-patterns with interference can be distinguished based on a threshold level Euclidean distance.

Based on the information provided in Table 2, an exemplary threshold level of 0.105 normalized units can be estimative to distinguish patterns and non-patterns with 100% sensitivity and specificity. Such exemplary pairings above 0.105 can indicate that the same pattern is not present on both signals, while pairings less than or equal to about 0.105 can indicate the same pattern being present on both signals. Using the exemplary threshold 0.105 for clustering and classification in, e.g., all 10 trials, the exemplary results are shown in Table 3, left-hand columns. For 10 trials, the sensitivity to correctly detect and distinguish patterns was 96.2%. The specificity to exclude non-patterns was 98.0%. For the test of interference+noise, a threshold value for TH2 of 0.132 was found to be efficacious in a test trial, and was then used in all trials. The exemplary results are shown in Table 3, right-hand columns, with mean values of about 89.1% for sensitivity and about 97.0% for specificity. Thus, the exemplary embodiment of the technique, method and system according to the present disclosure can be nearly as efficacious for classification when random noise as well as interference is added to CFAE.

TABLE 2

| Pattern | A | n | A | B | A | A | A | B | B | n | B |
|---|---|---|---|---|---|---|---|---|---|---|---|
| A | 0.000 | 0.142 | 0.056 | 0.133 | 0.092 | 0.074 | 0.065 | 0.128 | 0.135 | 0.221 | 0.135 |
| n | 0.142 | 0.000 | 0.151 | 0.149 | 0.166 | 0.123 | 0.131 | 0.188 | 0.156 | 0.143 | 0.122 |
| A | 0.056 | 0.151 | 0.000 | 0.136 | 0.097 | 0.068 | 0.068 | 0.143 | 0.145 | 0.214 | 0.138 |
| B | 0.133 | 0.149 | 0.136 | 0.000 | 0.167 | 0.127 | 0.144 | 0.101 | 0.096 | 0.185 | 0.063 |
| A | 0.092 | 0.166 | 0.097 | 0.167 | 0.000 | 0.095 | 0.101 | 0.196 | 0.206 | 0.271 | 0.151 |
| A | 0.074 | 0.123 | 0.068 | 0.127 | 0.095 | 0.000 | 0.082 | 0.156 | 0.124 | 0.191 | 0.116 |
| A | 0.065 | 0.131 | 0.068 | 0.144 | 0.101 | 0.082 | 0.000 | 0.156 | 0.151 | 0.212 | 0.152 |
| B | 0.128 | 0.188 | 0.143 | 0.101 | 0.196 | 0.156 | 0.156 | 0.000 | 0.105 | 0.241 | 0.102 |
| B | 0.135 | 0.156 | 0.145 | 0.096 | 0.206 | 0.124 | 0.151 | 0.105 | 0.000 | 0.161 | 0.104 |
| n | 0.221 | 0.143 | 0.214 | 0.185 | 0.271 | 0.191 | 0.212 | 0.241 | 0.161 | 0.000 | 0.168 |
| B | 0.135 | 0.122 | 0.138 | 0.063 | 0.151 | 0.116 | 0.152 | 0.102 | 0.104 | 0.168 | 0.000 |

A—pattern A.
B—pattern B.
n—nonpattern.
There is symmetry about the main diagonal.

TABLE 3

| trial # | sen: int | spe: int | sen: int + n | spe: int + n |
|---|---|---|---|---|
| 1 | 100.0 | 100.0 | 91.1 | 100.0 |
| 2 | 97.8 | 100.0 | 97.8 | 95.0 |
| 3 | 93.3 | 100.0 | 82.2 | 100.0 |
| 4 | 95.6 | 100.0 | 88.9 | 100.0 |
| 5 | 93.3 | 100.0 | 88.9 | 100.0 |
| 6 | 91.1 | 80.0 | 88.9 | 75.0 |
| 7 | 95.6 | 100.0 | 88.9 | 100.0 |
| 8 | 97.8 | 100.0 | 91.1 | 100.0 |
| 9 | 97.8 | 100.0 | 84.4 | 100.0 |
| 10 | 100.0 | 100.0 | 88.9 | 100.0 |
| mean | 96.2 ± 3.0 | 98.0 ± 6.3 | 89.1 ± 4.1 | 97.0 ± 7.9 |

sen—sensitivity,
spe—specificity,
int—interference,
n—noise

### Exemplary Spectral Profiles of Complex Fractionated Atrial Electrograms

For example, no significant changes occurred in any parameter from the first to second recording sequence. For both exemplary sequences, MPS and SPS were significantly greater, and DF and ADF were significantly less, in paroxysmals versus persistents. The MPS and ADF measurements from ensemble spectra produced the most significant differences in paroxysmals versus persistents (e.g., P<0.0001). DF differences were less significant, which can be attributed to the relatively high variability of DF in paroxysmals. The MPS was correlated to the duration of uninterrupted persistent AF prior to electrophysiologic study (P=0.01), and to left atrial volume for all AF (P<0.05)

### Exemplary Discussion

According to certain exemplary embodiments of the present disclosure, for example, a data-driven transform can be provided for application to CFAE signals. The basis can be constructed, for example, from the ensemble averages of signal segments and can be found to be orthogonal except for small integer-multiple relationships. The power in each ensemble average can be equivalent to the projection of the signal onto the corresponding basis (e.g., exemplary Eq. (15)). The relationship of the ensemble spectrum to the autocorrelation spectrum and to the Fourier power spectrum can be shown. While the autocorrelation spectrum can be based on correlation at a single lag w, the ensemble and Fourier power spectra can be based on correlation at multiple lags w, 2w, . . . , nw. During construction of the ensemble spectrum, the autocorrelation function at lags can be averaged, as compared to the Fourier power spectrum which is typically a sinusoidal curve fitting of the autocorrelation function. Several tests can be used to compare the efficacy of the Fourier transform, versus transformation using ensemble averaging, for representation of CFAE signal components.

At several levels of additive noise and interference, the highest peaks in the ensemble spectrum can corresponded to the frequencies of three synthetic drivers with higher accuracy as compared to Fourier spectral analysis (e.g., p<0.001). Similarly, when random noise corrupted actual CFAE signals, the ensemble spectrum can be more accurate than Fourier in representation of the DF (e.g., p<0.01). The ensemble basis can be found to be useful for representation of the signal morphology of the three independent synthetic drivers. When only interference was added, the top three ranked basis vectors in order of greatest power can correspond to the independent driver morphology. When phase noise (jitter) was added, the top three ranked basis vectors can correspond to driver morphology, but with some smoothing. When a single low-power, short duration component was added as would simulate a distant driver, it can be evident as a distinct peak in all 216/216 ensemble averaging spectra but in only 82/216 Fourier spectra. Finally, when both Fourier and ensemble were used for reconstruction of actual CFAE signals, the ordered ensemble basis from 1-12 vectors can be more accurate as compared with Fourier for representation (e.g., p<0.001). Thus, it can be found that the exemplary transform can be more efficacious for representation of independent generator frequencies and CFAE morphologies as compared to the Fourier transform.

### Exemplary Computational and Mathematical Considerations

Although ensemble analysis can be robust to noise and jitter, to further reduce their affect on signal analysis, the inner product between the spectrum and a model can be used for gradual, adaptive update (see, e.g., Reference [25]) or alternatively, finite differences can be used for adaptation (see, e.g., Reference [26]). When computing and/or determining the DF of atrial fibrillation signals, variation by as much as 2.5 Hz can occur over a time interval of a few seconds; hence tracking with time-frequency methods may be required for accurate analysis (see, e.g., References [27, 28]). Since ensemble averaging can be a form of autocorrelation, a minimum sequence length of two cycles of the periodic signal can be needed for construction of the frequency spectrum (which would result in a very course estimation). To include low frequency activity to a lower limit of 2 Hz, as can be done in accordance with certain exemplary embodiments of the present disclosure, a window of at least 1000 ms (1 s) is preferably used. Any such measurement can be updated by shifting the analysis window, for example, by 100-150 ms steps, to describe the time-frequency evolution of the signal (see, e.g., Reference [29]).

To reduce error when any such short sequences are utilized for analysis, a model-based approach for update of the spectral profile can be implemented (see, e.g., Reference [30]). In a study, the DF computed by Fourier analysis was compared with the mean, median, and mode activation rate, as obtained by electrogram marking, to determine efficacy (see, e.g., Reference [10]). However, as stated in that study, DF does not typically specifically reflect activation rate and therefore is typically only an approximate measure, with a level of uncertainty. For this reason, adding artificial drivers at specific frequencies, as well as to analyzing the degradation of actual DFs in CFAE when random noise is introduced, can act as tests to compare the Fourier versus ensemble methods. In each of the exemplary tests of DF measurement, the highest peak in the spectral range can be selected as the DF. The more accurate selection of DF in presence of noise and interference by ensemble analysis may in part be due to increased spectral power in the fundamental frequency relative to sub- and superharmonics as compared with Fourier (see, e.g., Reference [18]).

Knowledge of the mechanisms for onset and maintenance of atrial fibrillation can be scant or limited due to the difficulty in quantitative assessment of the CFAE signal with the Fourier method, which can distort the signal during preprocessing and suffers from phase noise degradation of the estimate (see, e.g., Reference [31]). By devising a data-driven frequency transform, independent drivers can be successfully extracted and characterized by both frequency and morphologic measurements. The transform can be further developed for clinical use by activation mapping of the substrate during AF in patients, identifying independent sources in the maps (focal or reentrant), and determining the correspondence of these to the most important ensemble basis vectors and frequency components. It is believed that ablation lesions at these sources can best prevent reinduction of AF (see, e.g., Reference [5,6]). Simulations have suggested that sinusoidal electric fields may be important for excitation of cardiac tissue (see, e.g., Reference [32]). If such sinusoidal generators exist in nature, they can be efficiently represented by the Fourier transform, which is typically based upon sinusoidal components, but also by the ensemble basis, from which any such components can be readily reconstructed. Certain exemplary embodiments of the present disclosure can include retrospective analysis of nonsynchronous CFAE when comparing the ensemble averaging method with the Fourier transform.

Other clinical research may project the AF signals onto ensemble space using exemplary Eq. (15) for solution of two- and multiple-class problems. A plot of x^{T} a_{w} versus w can be a rendition of the ensemble power spectrum. One way to express differences between CFAE can be based on the difference in Euclidean distance in n ensemble space, where n can be given in exemplary Eq. (5) and can equal the number of points in the power spectrum. This can be computed as the square root of the sum of squares difference in corresponding points between power spectra. Suppose for example that many CFAE recordings can be obtained simultaneously from the left atrium. The ensemble spectrum of each can be compared with its nearest neighbors, with the difference in spectra for all neighbors averaged. If this is done for the CFAE, areas with small spectral difference may suggest presence of a driver and/or other homogeneous regions where spectral characteristics are similar, while areas with large spectral difference may suggest presence of substrate heterogeneity and/or boundary areas where multiple drivers compete. Another method of classification can be to sum all CFAE in a neighborhood region, compute the ensemble basis, project each CFAE onto the global basis, and cluster and classify according to the position of each point in n ensemble space. Elsewhere, ensemble spectra have been used to analyze the DF of ventricular tachyarrhythmias (see, e.g., Reference [29]) and to assess videocapsule endoscopy images for estimation of small bowel motility (see, e.g., Reference [33]), as described in further detail below. Thus, this exemplary new transform may have wider application for clinical data analysis.

### Exemplary Clinical Procedure and Data Acquisition—Videocapsule Endoscopy

Exemplary clinical data was collected implementing/utilizing certain exemplary embodiments of the present disclosure. Patients were evaluated, for example, at Columbia University Medical Center, New York. Retrospective videocapsule endoscopy data was, for example, obtained from ten celiac patients on a regular diet or within a few weeks of starting a gluten-free diet. In these patients the diagnostic biopsy, taken while on a regular diet, showed Marsh grade II-IIIC lesions. Informed consent was obtained prior to videocapsule endoscopy. Indications for this procedure included, for example, suspected celiac disease or Crohn's disease, iron deficient anemia, obscure bleeding, and chronic diarrhea. Patients had serology and biopsy-proven celiac disease. These patients were being subsequently evaluated by videocapsule endoscopy because they were considered to have complicated disease such as abdominal pain unexplained by previous evaluation. Exclusion criteria included, for example, patients under 18 years of age, those with a history of or suspected small bowel obstruction, dysphagia, presence of pacemaker or other electromedical implants, previous gastric or bowel surgery, serum IgA deficiency, pregnancy, and chronic NSAID use or occasional NSAIDs use during the previous month. Preferably, complete videocapsule endoscopy studies, reaching the colon, were used for analysis. The retrospective analysis of videocapsule endoscopy data was approved by the Internal Review Board at Columbia University Medical Center.

The PillCamSB2 videocapsule (e.g., Given Imaging, Yoqneam, Israel) can be utilized to obtain the small bowel images in the study groups. The system typically includes a recorder unit, battery pack, antenna lead set, recorder unit harness, battery charger, recorder unit cradle, and real-time viewer with cable. The capsule can acquire two digital frames per second and can be a single-use pill-size device (see, e.g., Reference [15A]). For each patient undergoing the procedure, abdominal leads were placed, for example, in the upper, mid, and lower abdomen, and a belt that contained the data recorder and a battery pack was affixed around the waist. The subjects swallowed the videocapsule, for example, with radio transmitter in the early morning with approximately 200 cc of water, after a 12 hour fast without bowel preparation. Subjects were allowed to drink water, for example, 2 hours after ingesting the capsule, and to eat a light meal after 4 hours. The recorder received radioed images that were transmitted, for example, by the videocapsule as it passed through the gastrointestinal tract. The capsule reached the caecum in the participants from which retrospective data was used in this study. The belt data recorder was then removed, and the data was downloaded, for example, to a dedicated computer workstation. Videos were reviewed and interpreted, for example, by an experienced gastroenterologist using the HIPAA-compliant PC-based workstation equipped with Given Imaging analysis software, that was also used to export videos for further analysis. For example, videoclips of 200 frames each acquired from the small intestine for each patient by the patients' physicians were analyzed retrospectively.

The retrospectively obtained patient videoclips were then transferred to a dedicated PC-type computer for quantitative analysis. From each RGB color videoclip, grayscale images (e.g., 256 brightness levels, 0=black, 255=white) with an image resolution of 576×576 pixels, were extracted, for example, using Matlab Ver. 7.7, 2008 (e.g., Mathworks, Natick Mass.). One sequence of 10-20 frames was extracted from each videoclip, for example, in which air bubbles and opaque extraluminal fluids were absent. Each sequence of N frames from 10-20 was repeated to form a series 200 frames long, typical for videoclip quantitative analyses. Thus the total number of repeating sequences of length N in the synthesized 200 frame series was 200/N. Additionally, a single frame from one celiac patient in which air bubbles was the dominant feature in the image, selected at random, was extracted for use as an extraneous image frame.

### Exemplary Improved Frequency Resolution for Characterization of CFAE

According to one exemplary embodiments of the present disclosure, a comparison was made between the ability to resolve two closely-spaced frequency components in the physiologic range of interest using Fourier power spectral analysis, versus a new technique that utilizes signal averaging. The exemplary synthesized closely spaced frequency components and two exemplary additive interferences were selected at random from a set of, e.g., 216 CFAE. The values for digital sampling rate (e.g., 977 Hz) and sequence length (e.g., N=8192, 8.4 s sequences) can be typical of those used for frequency analysis of CFAE obtained during clinical EP study. Tests were made in the range of about 3-10 Hz, the electrophysiologic range for evaluation of atrial electrical activity. From 105 tests, the mean resolving power of Fourier versus the new technique (e.g., about 0.29 Hz versus about 0.16 Hz; p<0.001), were higher than the theoretical values but in accord with the presence of large interferences that could act to mask the frequency components. In 13/105 trials, interference masked frequency components in the Fourier power spectrum. By comparison, this occurred in only e.g., 4/105 trials using the new technique. The error in estimating the synthesized components was ±0.023 Hz using Fourier versus ±0.009 Hz using the exemplary technique, system and method according to present disclosure (e.g., p<0.001).

The use of the exemplary embodiment of the technique, system and method according to present disclosure, compared to Fourier produced an improved frequency resolution and improved compression with less loss of resolution. For a given time data, the exemplary embodiment of the technique, system and method according to present disclosure can provide, e.g., double the frequency resolution, as compared to that that uses the Fourier transform. A decrease in the time so as to produce the same or similar frequency resolution using the exemplary technique, system and method according to present disclosure versus those using the Fourier transform provides a significant advantage in reducing and/or preventing errors that may occur over time. Exemplary procedures that use the exemplary technique, system and method according to present disclosure can be performed in less time, for example, decreasing the fluoroscopy radiation received by a patient.

### Exemplary Identification of Recurring Patterns in Fractionated Atrial Electrograms

According to certain exemplary embodiment of the present disclosure, it is possible utilize an exemplary transform to characterize recurring patterns in CFAE. First, e.g., ensemble averages can be computed from signal segments of length w, repeated for all w in the frequency range of interest. From each ensemble average, an exemplary orthogonal basis vector can be constructed by repeating the ensemble average of length w for the entire signal length N. The inner product between basis vector and original signal can produce a transform coefficient, which can be the signal power at that frequency. The exemplary power spectrum can be a plot of the entire series of transform coefficients versus frequency. Exemplary transform coefficients resulting from the inner product of one signal with the basis vectors of another signal can take on negative as well as positive values, and can have an average level near zero if the signals are uncorrelated. The correlation coefficients formed from correlated signal x with the basis vectors of z can be similar to the spectrum of x and is termed the spectral signature. Transform coefficients can be used to detect two recurring patterns in a sequence of CFAE, embedded in interference and random noise, and to distinguish them from each other and from non-patterns. For example, no manual intervention was used except to set initial threshold levels of Euclidean distance for identification of correlated content, i.e., for pattern extraction, and to distinguish the extracted patterns.

The spectral signature can be a graph of the correlated content between two signals in frequency space, which can be exploited for pattern recognition. If a series of signals is averaged and basis vectors of the mean are used to obtain the spectral signature of each individual signal, then there can be a correlation between the spectrum of the mean, and the spectral signature of the individual signal, when the individual signal contains a synchronous pattern that recurs within the series. By measuring the Euclidean distance between all individual signals having spectral signatures similar to the power spectrum of the mean signal, patterns contained in the sequence can be identified, distinguished from one another, and distinguished from non-patterns when the non-patterns are mostly uncorrelated with respect to the mean signal. Thus the exemplary technique, system and method according to present disclosure can be used to automatically identify and distinguish repetitive patterns present in a series of signals, once threshold levels for the Euclidean distance estimate to detect candidate patterns, and to discern patterns, are established. the determination of the exemplary patterns, if multiple patterns are present, the patterns can also be discerned using a single threshold level, since the Euclidean distance will be short only with respect to members of the same class. Successful source pattern recognition can be useful in catheter ablation as an identification of a source pattern provides for an area for best ablation.

### Exemplary Frequency Resolution for Characterization of CFAE

According to an exemplary embodiment of the present disclosure, by normalizing the CFAE spectra, it is possible to compare the CFAE frequency patterns observed in longstanding, persistent AF to those present in acutely induced AF in patients whose arrhythmia is clinically paroxysmal and whose baseline rhythm was sinus. Exemplary results indicate that the CFAE recordings during acute onset AF in patients with paroxysmal AF had significantly larger mean and standard deviation in the normalized power spectra, suggesting, but not proving, the presence of more randomly varying activation sources in general. By comparison, CFAE spectra from longstanding AF patients had lower mean value and standard deviation of spectral peaks, as would be expected if the peaks were generated by more stable and stationary sources present in the atrial substrate. The exemplary results also indicate that the CFAE recordings during acute onset AF in patients with paroxysmal AF had significantly lower amplitude and frequency of the dominant peak. This indicates a greater complexity in the power spectral profile of paroxysmal patients, which can likely be due to the presence of more peaks that are greatly varying in height, with no single predominant tall peak in the spectrum.

### Exemplary Image Corruption

Each of the exemplary image series was corrupted, for example, by the following exemplary methods (e.g., one or multiple used at the same time):

1. Temporal Phase Noise:

the 200 frame series was altered, for example, by removing 1-5 frames from the beginning or end of one of the repeating sequences comprising the series, and appending it to another of the sequences. This was done, for example, 2-3 times at random for each 200 frame series.

2. Spatial Phase Noise:

each image in the 200 frame series was altered, for example, using a maximum row-by-row pixel rotation of m=1-20 pixels. The degree of pixel rotation was the same for each row in a particular image, but was varied randomly from one image to the next from 0 to m.

3. Addition of Random Noise:

a series of X image frames were removed, for example, from the end of the 200 frame series and replaced with X white noise frames, where the number of frames removed was varied from X=0 to 180.

4. Addition of Bubble Image:

5-10 images were randomly removed, for example, from the 200 frame series and replaced with an image composed primarily of bubbles that did not belong in the series. The image used was the same for each of the ten 200 frame series that were analyzed.

The DP was calculated for each 200 frame series without any corruption, and with imposition of one or more of the methods listed above (total of 20 trials for each series).

### Exemplary Spectral Analysis

Both the Fourier and exemplary ensemble spectral analysis methods can be used for DP calculation. For analysis, the series of 200 grayscale brightness values at each pixel location can be treated, for example, as a signal. Each of these 576×576=331776 signals can be, for example, first set to mean zero. Then, the power spectrum (e.g., Fourier or ensemble method) can be computed for each, and the average of all 331776 individual power spectra can be, for example, considered to be the videocapsule frequency spectrum. The tallest peak in the power spectrum can be taken as the DF, which is related to the DP, based on a frame rate of 2 per second, as, e.g.:

DP=2./DF (22)

where DF can have units of Hz and DP can have units of seconds. All computation can be done, for example, using a Lenovo x60 laptop computer, Windows XP Pro (Service Pack 3) operating system, and Intel T2400 processor running at 1.83 GHz with 3 GB of RAM memory.

Prior to Fourier spectral calculation, the exemplary 200 point data array can be smoothed using a Hann window of the form, e.g.:

a[k+1]=0.5*[1−cos(2πk/(n−1)],k=0,1, . . . n−1 (23)

where a[k] can be, for example, the weights by which the 200 point array are multiplied. The windowed data can be then padded with 56 zeros to form an array 28=256 points. Since the sample rate was 2 frames/second, e.g.:

$$\begin{matrix}resolution = sample rate / signal length N \\ = ( 2 frames \text{/} s ) / 256 frames\end{matrix} \qquad ( 24 )$$

which is, for example, 0.0078 Hz. The Fast Fourier Transform (FFT) can be computed using the Intel Visual Fortran Compiler 9.0 Build Environment for 32-bit applications (Intel Corporation, 2005) using the subroutine ‘four1’ provided by Numerical Recipes in Fortran 77 (see, e.g., Reference [16A]). This radix-2 implementation, which can apply to real data arrays of length 2N, is used in the literature, although it is not the most efficient FFT code (see, e.g., Reference [17A]). The Fourier power spectrum can be computed, for example, as the magnitude of the real and imaginary parts of the FFT as computed in double-precision mode, and plotted versus frequency.

The ensemble average method of spectral analysis has been described elsewhere (see, e.g., References [6A, 7A]). In short, the ensemble average vector e_{w}, for example, can be obtained by averaging successive mean-zero signal segments of length w, e.g.:

e_{w}=1/n·U_{w}·b (25a)

U_{w}=[I_{w} I_{w} . . . I_{w}] (25b)

where b can be, for example, the signal vector of length N and I_{w} can be w×w identity submatrices used, for example, to form the signal segments that are extracted from x and summed. The pixel brightness signals b were not windowed or otherwise filtered prior to analysis using ensemble averaging. The number of signal segments of window length w being summed can be, e.g.:

n=int(N/w) (26)

where int is typically needed if n·w≠N. The power in the ensemble average is given by, e.g.:

P_{w}=1/we_{w}^{T}·e_{w} (27)

To generate an ensemble power spectrum, the root mean square (RMS) power can be utilized to reduce the affect of outliers (see, e.g., References [6A, 7A]):

PwRMS=sqrt(Pw) (28)

where sqrt can be, for example, the square root function and the units are millivolts. The power spectrum can be then formed by plotting sqrt(n)×PwRMS versus frequency f, where, e.g.:

f=sample rate/w (29)

The sqrt(n) term levels the noise floor, which can be otherwise diminish by 1/sqrt(n), the falloff per number of summations n used for ensemble averaging. As an additional device to level the noise floor, the linear regression line can be calculated from the graph points and then subtracted from these points. For simplicity, the ensemble average spectrum can be computed using integer values of w, resulting in higher resolution at lower frequencies and lower resolution at higher frequencies due to the relationship of exemplary Eq. (29). If, however, fractional values of w were to be used with interpolation between data points, the ensemble average frequency spectrum can be made uniform in resolution.

The same Fortran compiler that was used for Fourier analysis can be also utilized to calculate or determine the ensemble average spectrum (single precision mode). Previously, the DP has been observed to occur in the range 1-20 seconds (see, e.g., Reference [8A]). Thus, for both the Fourier and ensemble averaging methods, the spectral range can be selected as, for example, 0.05 Hz (period=20 seconds) to 1 Hz (period=1 second).

### Exemplary Computational Considerations

Typical FFT procedures generally require log N·N operations to complete. For the radix-2 implementation, the 200 frames padded to 2n=256 typically require approximately 2.4·256=614 operations to calculate. By comparison, the ensemble procedure can use the number of frames that are available (e.g., 200 in this case). Its spectrum can range from highest frequency (segment length w=2) to lowest frequency (w N/2). To detect the DP in the range 1-20 s, segment lengths from w=2 (period 1 s) to 40 (period 20 s) can be used as endpoints for spectral analysis. For spectral power computation, e.g.:

$$\begin{matrix}\# operations \approx [ {\sum }_{i} ( ni - 1 ) * w i ] + wi , i = x to y & ( 30 a ) \\ = {\sum }_{i} N , i = x to y & ( 30 b ) \\ = ( y - x ) \cdot N & ( 30 c ) \\ = c \cdot N & ( 30 d )\end{matrix}$$

where the value of n is obtained from Eq. (26), x and y are the spectral endpoints, c=y−x is the number of frequency components computed per spectrum, and the +wi term on the RHS in Eq. (30a) is due to the sum of squares divided by w calculation which determines the ensemble average power Eq. (27). Since the ensemble average for segment length wi=wj/2 can be computed as:

e_{wi}=e_{wj}(1:wⱼ/2)+e_{wj}(wⱼ/2+1:wⱼ) (31)

The number of operations to compute or determine the ensemble spectrum can be readily reduced from c·N (exemplary Eq. (30d)) to c/2·N using exemplary Eq. (31). For x=2 and y=40, c=39, so that for N=200 frames, 7800 operations are typically needed to compute an ensemble spectrum, as compared with 614 for Fourier. However, most of the ensemble average operations are simple addition. Thus, without testing it is typically not apparent whether the Fourier or ensemble average method will be faster to compute the power spectra. Speed can be an important consideration when many videoclips from many patients, and/or longer series lengths than 200, are used for analysis in future studies. As a test of computational speed, the Fourier versus ensemble spectral calculation over 576×576=331776 pixels can be determined, for example, by using the internal Fortran function ‘etime’ (user elapsed time), which can be printed on the computer screen during program execution. The difference in the elapsed program run time at start versus end of the 576×576 pixel spectra routine can be taken as the spectral computation time. For faster Fourier computation, the variables can be computed in single-rather than double-precision mode in the speed calculations, which can be observed to reduce computation time by about 10% without evident quantitative effect on Fourier spectral calculation. Speed measurement can be repeated 10 times each for Fourier and ensemble, with pauses of several minutes in between to allow the computer to return to its quiescent state.

### Exemplary Results

The exemplary result from a repeating 10 frame sequence is shown, for example, in the exemplary images of FIGS. 12A-12F to highlight how additive noise affects the images. Each panel shows the result of ensemble averaging every 10th frame from a 200 frame synthesized series (frames 1, 11, . . . , 191). Since the repeating sequence can be, for example, 10 frames long, this can result in correlation in the ensemble average. In panel A, the result is depicted, for example, when the exemplary 200 frame series lacked any additive noise. Thus, the average in panel of FIG. 12A, for example, is simply the first image frame in the 10 frame sequence that was used for synthesis. The detail in the image of panel A includes presence of numerous mucosal folds, small mucosal surface abnormalities, and extraneous substances. This can be a typical image from celiac videoclips taken from the distal duodenum. Subsequently, noise was imparted to the same synthesized 200 frame series. When a random temporal shift of 1-5 frames was imposed on the series, the resulting averaged image from frames 1, 11, . . . , 191 is shown, for example, in panels of FIGS. 12B-12F, respectively. Thus, in panel B there is, for example, a random temporal phase shift of ±1 frame in the 200 frame sequence. The frame shift can be increased in each successive panel, thus in frame of FIG. 12F, there can be a random temporal shift of ±5 frames in the 200 frame sequence. As a result of larger phase shifts, the images in panels of FIGS. 12D-12F can have partially visually morphed to resemble other images in the 10 frame sequence. However, features from the original image having marked contrast as compared to the background can be retained—for example at the asterisks in each frame. Additionally, an imposition of three air bubble frames in the series that was averaged (1, 11, . . . , 191) is shown, for example, in panel F. Air bubbles can be clearly evident within the image, particularly in the lower right quadrant, which contributes to the noise level. Spectral analysis of the series can be expected to readily detect the 10 frame period in the series when no noise was imparted (e.g., panel of FIG. 12A), but to have progressively more difficulty as extraneous features were imposed (e.g., panels of FIGS. 12B-12F).

According to certain further exemplary embodiments of the present disclosure, two additional types of image degradation that can be imposed on the substantially same videoclip series as is shown in FIGS. 12A-12F are noted in FIGS. 13A-13F, respectively. For example, panel of FIG. 13A is substantially the same as the corresponding panel in FIG. 12A, and again is an average of, for example, the 1st 11th, . . . , 191st frames in the synthesized videoclip series. Since the synthesized series included, for example, a 10 frame sequence that was repeated, panel A is also the first frame in that sequence. The panels of FIGS. 13B-13F were formed after imposing a maximum spatial frame shift, for example, of 20 pixels on the 200 frame series. As described herein, each row of pixels in the original images used in the series can be rotated by up to 20 pixels per image. The same procedure and magnitude of spatial frame shift can be imposed in all panels of FIGS. 13B-13F, Additionally, from panels B-F there can be added a successively increased random noise content, for example, with X=10, 50, 100, 150, and 180 frames at the end of the 200 frame series being switched to white noise frames, respectively. Thus, FIG. 13B can be constructed with only one noise frame of the 20 frames 1, 11, . . . , 191 used for ensemble averaging, panel of FIG. 13C with 5, of FIG. 13D with 10, of FIG. 13E with 15, and of FIG. 13F with 18 noise frames out of 20 frames. It can be evident that panel F only slightly resembles the original image depicted, for example, in panel of FIG. 13A.

An example of spectral analysis using the Fourier method is shown, for example, in FIG. 14, again for the case of a 10 frame repetitive sequence for continuity with FIGS. 12A-12F and 13A-13F (e.g., DP=5 s, DF=0.2 Hz). To the 200 frame series, random temporal shift, for example, of up to +4 frames was imparted and random noise was added. The number of random noise frames out of 200 total frames used for analysis is shown at upper left in each panel. Even in the case of no additive random noise, and only temporal frame shift imposed on the series, the harmonic peaks show a drastic split (e.g., see graph of FIG. 14A). At the 0.2 Hz mark, there can be a small peak present—the split peaks can be off-center. Furthermore, peaks of greater energy can occur, for example, at 0.4 Hz and 0.6 Hz. Thus, this spectrum may not be used to correctly determine the DP. Similar spectral features are present in panel B, when, for example, 50 random white noise frames can be switched into the 200 frame series. In panel C, the increased additive random noise at the level of 100 frames, half the series, can have a smoothing effect on the spectral peaks. Still, the peak near 0.2 Hz can be substantially off-center, with the peaks at 0.4 Hz and 0.6 Hz, for example, being of greater energy. At the highest additive random white noise level (e.g., panel of FIG. 14D), all of the harmonics can shift off-center in different directions, they become blunted, and the peak near 0.2 Hz can be further eroded. Thus, there can be difficulty in using the Fourier method for analysis of this videocapsule frame series for DP determination at the imposed level of noise degradation.

Analysis of the same 200 frame series with the same additive noise and temporal phase shift levels as depicted in FIG. 14 is shown in FIG. 15, when the ensemble average procedure was used, for example, for spectral analysis. In each panel, the dominant peak occurs, for example, at 0.2 Hz. Subharmonics and the superharmonic at 0.4 Hz can be evident but they can be lesser in value. There can be little difference in the detail in each spectrum, i.e., the procedure can be robust to varying, even overwhelming, levels of additive noise. Therefore, in each panel, the DF at 0.2 Hz (DP of 5 s), for example, can be correctly identified. Because of the f=1/w relationship (see exemplary Eq. (28)) the frequency resolution is not typically uniform; however, the detection of DP can be unaffected.

Fourier spectra when all four types of image degradation can be added to the 200 frame series (see above) are shown, for example, in FIG. 16. Again for ease of comparison with the other figures, in this exemplary series, a 10 frame repeating sequence can be also used (e.g., DF=0.2 Hz, DP=5 s). The series can have both spatial phase noise (e.g., ±10 pixels) and temporal phase noise (e.g., ±3 frames) imparted as well as additive random noise, and eight air bubble frames can be switched in. As shown in the graphs of in FIGS. 14A-14D and 15A-15D, the number of random noise frames is shown, for example, at upper left in each panel. As in FIG. 14A, the harmonic peaks can be split when no random noise is added (see FIG. 16A). The peak with greatest energy can occur at, for example, 0.17 Hz, and the harmonic peaks can be of lesser magnitude. The tallest peak can be maintained at random noise levels of for example, 50 and 100 frames (e.g., panels of FIGS. 14B, 15B, 16B and 14C, 15C, 16C). However at the highest additive random noise level shown (e.g., 180, panel of FIG. 16D) the dominant peak has typically been completely corrupted so that the new dominant peak occurs at, for example, 0.09 Hz, barely above the noise floor. Thus, in this example as in FIGS. 14A-14D, Fourier spectra may not be accurate for pinpointing the DP.

Spectra created using the exemplary ensemble average method are shown, for example, in FIG. 17 for the substantially same data as those from which FIG. 16 was constructed. As in the graphs of FIGS. 15A-15D ensemble average spectra, the ensemble average spectra of FIGS. 17A-17D, respectively, correctly depict, for example, 0.2 Hz as the DF. There is no shift or corruption of the dominant peak, for example, even at the highest additive random noise level of 180 frames. At the 180 noise frame level, only 20 frames can be actual signal (10%), less those frames for which the air bubble frame was switched in. There can be however, a slight broadening of some dominant peaks (e.g., see FIGS. 17B and 17D).

### Exemplary Summary Statistics

As is shown in Table 4, for all additive noise levels, the mean absolute difference between estimated versus actual DP can be, for example, 0.0547±0.0688 Hz for Fourier versus 0.0031+0.0127 Hz for ensemble (e.g., p<0.001 in mean and standard deviation). The mean time for computing 331,776 pixel spectra per videoclip can be, for example, 12.31±0.01 s for Fourier versus 4.86±0.01 s for ensemble (p<0.001).

TABLE 4

| | | Ensem- | Signi- | | Ensem- | Signi- |
|---|---|---|---|---|---|---|
| Statistic | Fourier* | ble* | ficance | Fourier\hat{ } | ble\hat{ } | ficance |
| MN | 0.0547 | 0.0031 | p < 0.001 | 12.31 | 4.86 | p < 0.001 |
| SD | 0.0668 | 0.0127 | p < 0.001 | 0.01 | 0.01 | MS |

According to additional exemplary embodiments of the present disclosure, pixel spectral analysis for videocapsule image quantization can be provided. Certain exemplary embodiments can show that even in presence of overwhelming noise and extraneous features imposed upon small intestinal mucosal image series, examples of which are shown in FIGS. 12 and 13, the exemplary pixel-by-pixel procedure of frequency analysis can be useful to detect the DP when ensemble averaging is used for computation. Additionally, the exemplary ensemble average calculation has advantage of speed over Fourier analysis using the computer described above. In previous work, the average brightness of the entire image frame, for 200 videoclip frames, was for example, used as inputs for spectral analysis (see, e.g., Reference [18A]), e.g.:

b=<b1>,<b2>, . . . , <b200> (32)

where b can be the input for spectral analysis and <•> denotes the frame average brightness, frames 1-200. This simpler method was found useful to find a significant DP difference in celiac versus control videoclips (e.g., longer DP in celiacs). Yet, the exemplary pixel-by-pixel spectral calculation, followed by averaging to form the mean spectrum, is potentially more efficacious for detecting subtle periodicities in videocapsule images because more information can be accounted for.

### Exemplary Analysis of Videocapsule Endoscopy Images

Endoscopy of the small intestine can be helpful for detecting villous atrophy, a common manifestation of untreated celiac disease, although this is typically confirmed by biopsy (see, e.g., Reference [19A]). The typical treatment for celiac disease currently available that can restore the intestinal villi and also eliminate systemic symptoms of the disease, is a lifelong gluten-free diet (see, e.g., References [9A, 19A]). However, months on the diet are typically needed to substantially restore the small intestinal villi, and in some patients only partial restoration occurs or there may be no restoration. Among prior quantitative analysis studies of the small intestine to detect villous atrophy, duodenal features have been classified using Fourier filters in magnifying endoscopic images (see, e.g., Reference [20A]). Yet, some intestinal regions lack visible change while villous atrophy can be present, which can diminish the sensitivity of the classification method. The textural properties of images from the small intestinal mucosa in celiac disease has been investigated (see, e.g., Reference [18A]).

The variance in grayscale brightness can be used as an estimate of texture. Over 200 image frame series in celiac versus control videocapsule studies, the celiacs typically had significantly greater texture magnitude even in distal portions of the small intestine (jejunum and ileum). This suggested the possibility that villous atrophy can be widespread in the intestinal lumen in untreated celiac patients, but may be below the threshold for visual detection by eye. Quantitative parameterization over 200 sequential images would therefore be expected to have merit for analysis of small intestinal pathology in these patients. Yet, the textural procedure can be sensitive to ambient conditions including changing camera angle with respect to the luminal wall, and to illumination (see, e.g., References [8A, 18A]). Hence, more recently using frequency analysis over 200 frames, which would be anticipated to be sensitive to periodic oscillations in frame-to-frame brightness variation due to small intestinal motility. It can be supposed that the method would be robust to ambient conditions like camera angle and illumination, as the oscillations would be typically reflected in the frequency content while changes in ambient conditions would mostly just affect the overall spectral power (see, e.g., Reference [8A]). Exemplary embodiments of the present disclosure can provide evidence that the DP can be in fact an important repeating pattern in 200 frame series, where importance can be synonymous with having the greatest spectral power, and that pixel-by-pixel spectra calculation can be robust to even large-scale extraneous features.

Although descriptions of certain exemplary embodiments of the present disclosure have been limited, for simplicity, to converting the color videocapsule images to 256 level grayscale for quantitative analysis, abnormal patterns can also be detected in color space using nonlinear methods (see, e.g., Reference [21A]). Here, the nonlinear approach was used to detect specific features—in this case ulcerous regions versus normal mucous membrane in the small intestine. Their analysis showed that the green component of RGB can contain the bulk of the ulcer information, with classification accuracy exceeding 95.5%. Although small intestine villi can be much more subtle in structure than are ulcerous regions, the use of a specific color (e.g., green, red, or blue) rather than grayscale may be useful to improve the exemplary procedures for frequency detection.

### Exemplary Motility Measurement in Videocapsule Endoscopy

Although videocapsule endoscopy has been commercially available for approximately 10 years (see, e.g., Reference [22A]), the images can be presently used by the gastroenterologist typically as a qualitative assist device when assessing the extent and severity of villous atrophy (see, e.g., References [23A-25A]). Gastrointestinal motility is also likely altered in untreated celiac disease due to injury to the mucosa, but is typically only indirectly gauged, by measuring the transit time from proximal to distal small intestine. To establish a more direct link between the mechanical characteristics of the small intestine and celiac disease, the exemplary frame-by-frame frequency analysis has been proposed, and in a prior study found a direct correlation between transit time and DP (see, e.g., Reference [8A]).

FIG. 18 shows a block diagram of an exemplary embodiment of a system according to the present disclosure. For example, exemplary procedures in accordance with the present disclosure described herein can be performed by a processing arrangement and/or a computing arrangement 102. Such processing/computing arrangement 102 can be, e.g., entirely or a part of, or include, but not limited to, a computer/processor 104 that can include, e.g., one or more microprocessors, and use instructions stored on a computer-accessible medium (e.g., RAM, ROM, hard drive, or other storage device).

As shown in FIG. 18, e.g., a computer-accessible medium 106 (e.g., as described herein above, a storage device such as a hard disk, floppy disk, memory stick, CD-ROM, RAM, ROM, etc., or a collection thereof) can be provided (e.g., in communication with the processing arrangement 102). The computer-accessible medium 106 can contain executable instructions 108 thereon. In addition or alternatively, a storage arrangement 110 can be provided separately from the computer-accessible medium 106, which can provide the instructions to the processing arrangement 102 so as to configure the processing arrangement to execute certain exemplary procedures, processes and methods, as described herein above, for example.

Further, the exemplary processing arrangement 102 can be provided with or include an input/output arrangement 114, which can include, e.g., a wired network, a wireless network, the internet, an intranet, a data collection probe, a sensor, etc. As shown in FIG. 18, the exemplary processing arrangement 102 can be in communication with an exemplary display arrangement 112, which, according to certain exemplary embodiments of the present disclosure, can be a touch-screen configured for inputting information to the processing arrangement in addition to outputting information from the processing arrangement, for example. Further, the exemplary display 112 and/or a storage arrangement 110 can be used to display and/or store data in a user-accessible format and/or user-readable format.

The foregoing merely illustrates the principles of the disclosure. Various modifications and alterations to the described embodiments will be apparent to those skilled in the art in view of the teachings herein. It will thus be appreciated that those skilled in the art will be able to devise numerous systems, arrangements, and procedures which, although not explicitly shown or described herein, embody the principles of the disclosure and can be thus within the spirit and scope of the disclosure, including using the features and various exemplary embodiments described herein together and interchangeably with one another. In addition, all publications and references referred to above can be incorporated herein by reference in their entireties. It should be understood that the exemplary procedures described herein can be stored on any computer accessible medium, including a hard drive, RAM, ROM, removable disks, CD-ROM, memory sticks, etc., and executed by a processing arrangement and/or computing arrangement which can be and/or include a hardware processors, microprocessor, mini, macro, mainframe, etc., including a plurality and/or combination thereof. In addition, certain terms used in the present disclosure, including the specification, drawings and claims thereof, can be used synonymously in certain instances, including, but not limited to, e.g., data and information. It should be understood that, while these words, and/or other words that can be synonymous to one another, can be used synonymously herein, that there can be instances when such words can be intended to not be used synonymously. Further, to the extent that the prior art knowledge has not been explicitly incorporated by reference herein above, it is explicitly incorporated herein in its entirety. All publications referenced are incorporated herein by reference in their entireties.

### EXEMPLARY REFERENCES

1. Ciaccio E J, Dunn S M, Akay M, Wit A L, Coromilas J, Costeas C A. Localized spatial discrimination of epicardial conduction paths after linear transformation of variant information. Ann Biomed Eng. 1994, 22:480-492.
2. Mandapati R, Skanes A, Chen J, Berenfeld O, Jalife J. Stable microreentrant sources as a mechanism of atrial fibrillation in the isolated sheep heart. Circulation. 2000, 101:194-199.
3. Nademanee K, McKenzie J, Kosar E, Schwab M, Sunsaneewitayakul B, Vasavakul T, Khunnawat C, Ngarmukos T: A new approach for catheter ablation of atrial fibrillation: Mapping of the electrophysiologic substrate. J Am Coll Cardiol 2004, 43:2044-2053.
4. Oral H, Chugh A, Good E, Sankaran S, Reich S S, Igic P, Elmouchi D, Tschopp D, Crawford T, Dey S, Wimmer A, Lemola K, Jongnarangsin K, Bogun F, Pelosi F Jr, Morady F. A tailored approach to catheter ablation of paroxysmal atrial fibrillation. Circulation. 2006 18, 113:1824-1831.
5. Niu G, Scherlag B, Lu Z, Ghias M, Zhang Y, Patterson E, Dasari T, Zacharias S, Lazzara R, Jackman W M, Po S S. An acute experimental model demonstrating two different forms of sustained atrial tachyarrhythmias. Circ Arrhythmia Electrophysiol. 2009, 2:384-392.
6. Kapa S, Asirvatham S J. Atrial Fibrillation: Focal or Reentrant or Both? Circulation: Arrhythmia and Electrophysiology 2009, 2:345-348.
7. Sanders P, Berenfeld O, Hocini M, Jais P, Vaidyanathan R, Hsu L F, Garrigue S, Takahashi Y, Rotter M, Sacher F, Scavee C, Ploutz-Snyder R, Jalife J, Haïssaguerre M. Spectral analysis identifies sites of highfrequency activity maintaining atrial fibrillation in humans. Circulation 2005, 112:789-797.
8. Botteron G W, Smith J M: A technique for measurement of the extent of spatial organization of atrial activation during atrial fibrillation in the intact human heart. IEEE Trans Biomed Eng 1995, 42:579-586.
9. Botteron G W, Smith J M. Quantitative assessment of the spatial organization of atrial fibrillation in the intact human heart. Circulation 1996, 93:513-518.
10. Ng J, Kadish A H, Goldberger J J. Effect of electrogram characteristics on the relationship of dominant frequency to atrial activation rate in atrial fibrillation. Heart Rhythm 2006, 3:1295-1305.
11. Ng J, Kadish A H, Goldberger J J. Technical considerations for dominant frequency analysis. J Cardiovasc Electrophysiol 2007, 18:757-764.
12. Ng J, Goldberger J J. Understanding and interpreting dominant frequency analysis of electrograms. J Cardiovasc Electrophysiol 2007, 18:680-685.
13. Fischer G, Stühlinger M C, Nowak C N, Wieser L, Tilg B, Hintringer F. On computing dominant frequency from bipolar intracardiac electrograms. IEEE Trans Biomed Eng 2007, 54:165-169.
14. Verma A, Patel D, Famey T, et al. Efficacy of adjuvant anterior left atrial ablation during intracardiac echocardiography-guided pulmonary vein antrum isolation for atrial fibrillation. J Cardiovasc Electrophysiol 2007, 18:151-6.
15. Brooks A G, Stiles M K, Laborderie J, Lau D H, Kuklik P, Shipp N J, Hsu L F, Sanders P. Outcomes of long-standing persistent atrial fibrillation ablation: A systematic review. Heart Rhythm 2010, 7:835-846.
16. Ciaccio E J. Ablation of Longstanding Persistent Atrial Fibrillation. Journal of Atrial Fibrillation 2010, 2:806-809.
17. Ciaccio E J, Biviano A B, Whang W, Wit A L, Garan H, Coromilas J. New methods for estimating local electrical activation rate during atrial fibrillation. Heart Rhythm 2009, 6:21-32.
18. Ciaccio E J, Biviano A B, Whang W, Wit A L, Coromilas J, Garan H. Optimized measurement of activation rate at left atrial sites with complex fractionated electrograms during atrial fibrillation. J Cardiovasc Electrophysiol, 2010, 21:133-143.
19. Narayan S M, Krummen D E, Kahn A M, Karasik P L, Franz M R. Evaluating fluctuations in human atrial fibrillatory cycle length using monophasic action potentials. PACE 2006, 29:1209-1218.
20. Brown J P, Krummen D E, Feld G K, Narayan S M. Using electrocardiographic activation time and diastolic intervals to separate focal from macro-re-entrant atrial tachycardias. J Am Coll Cardiol 2007, 49:1965-1973.
21. Narayan S M, Franz M R. Quantifying fractionation and rate in human atrial fibrillation using monophasic action potentials: Implications for substrate mapping. Europace 2007, 9:vi89-vi95.
22. Stiles M K, Brooks A G, John B; Shashidhar, Wilson L, Kuklik P, Dimitri H, Lau D H, Roberts-Thomson R L, Mackenzie L, Willoughby S, Young G D, Sanders P. The effect of electrogram duration on quantification of complex fractionated atrial electrograms and dominant frequency. J Cardiovasc Electrophysiol. 2008; 19:252-258.
23. Biviano A B, Coromilas J, Ciaccio E J, Whang W, Hickey K, Garan H. Frequency Domain and Time Complex Analyses Manifest Low Correlation and Temporal Variability When Calculating Activation Rates in Atrial Fibrillation Patients. Pacing Clin Electrophysiol. 2011 Jan. 5. doi: 10.1111/j.1540-8159.2010.02993.x. [Epub ahead of print]
24. MacLeod R S, Johnson C R. Map3d: Interactive scientific visualization for bioengineering data. IEEE Engineering Medicine Biology Society 15th Ann Int Conference 1993:30-31.
25. Stridh M, Sörnmo L, Meurling C J, Olsson S B. Sequential characterization of atrial tachyarrhythmias based on ECG time-frequency analysis. IEEE Trans Biomed Eng. 2004, 51:100-114.
26. Ciaccio E J, Drzewiecki G M. Tonometric arterial pulse sensor with noise cancellation. IEEE Trans Biomed Eng. 2008, 55:2388-2396.
27. Stridh M, Bollmann A, Olsson B, Sörnmo L. Detection and feature extraction of atrial tachyarrhythmias. A three stage method of time-frequency analysis. IEEE Engineering Medicine Biology Magazine 2006, 25:31-39.
28. Sandberg F, Stridh M, Sörnmo L. Frequency tracking of atrial fibrillation using hidden markov models. IEEE Trans Biomed Eng 2008, 55:502-511.
29. Ciaccio E J, Coromilas J, Wit A L, Garan H. Onset dynamics of ventricular tachyarrhythmias as measured by dominant frequency. Heart Rhythm 2011 doi:10.1016/j.hrthm.2010.11.028 (in press).
30. Corino V D A, Mainardi L T, Stridh M, Sörnmo L. Improved time-frequency analysis of atrial fibrillation signals using spectral modeling. IEEE Trans Biomed Eng. 2008, 55:2723-2730.
31. Elvan A, Linnenbank A C, van Bemmel M W, Ramdat Misier A R, Delnoy P P H M, Beukema W P, de Bakker J M T. Dominant frequency of atrial fibrillation correlates poorly with atrial fibrillation cycle length. Circulation: AE 2009, 2:634-644.
32. Gray R A, Mornev O A, Jalife J, Aslanidi O V, Pertsov A M. Standing excitation waves in the heart induced by strong alternating electric fields. Phys. Rev. Lett. 2001, 87:168104-1-168104-4.
33. Ciaccio E J, Tennyson C A, Lewis S K, Bhagat G, Green P H. Distinguishing patients with celiac disease by quantitative analysis of videocapsule endoscopy images. Compute Methods Programs Biomed 2010, 100:39-48.
34. Ciaccio E J, Biviano A B, Whang W, Vest J A, Gambhir A, Einstein A J, Garan H. Differences in repeating patterns of complex fractionated left atrial electrograms in longstanding persistent as compared with paroxysmal atrial fibrillation. Circulation: Arrhythmia and Electrophysiology (accepted) 2011.
35. Ciaccio E J, Biviano A B, Whang W, Garan H. Improved Frequency Resolution For Characterization Of Complex Fractionated Atrial Electrograms Frequency Resolution Of Atrial Electrograms.
36. Ciaccio E J, Biviano A B, Whang W, Garan H. Identification Of Recurring Patterns In Fractionated Atrial Electrograms Using New Transform Coefficients. Biomedical Engineering OnLine 2012.
37. Ciaccio E J, Biviano A B, Whang W, Gambhir A, Einstein A J, Garan H. Spectral Profiles Of Complex Fractionated Atrial Electrograms Are Different In Longstanding And Acute Onset Atrial Fibrilation Atrial Electrogram Spectra. Aptara 2012.
[1A] Botteron G W, Smith J M: A technique for measurement of the extent of spatial organization of atrial activation during atrial fibrillation in the intact human heart. IEEE Trans Biomed Eng 1995; 42:579-586.
[2A] Botteron G W, Smith J M: Quantitative assessment of the spatial organization of atrial fibrillation in the intact human heart. Circulation 1996; 93:513-518.
[3A] Fischer G, Stühlinger M C, Nowak C N, Wieser L, Tilg B, Hintringer F: On computing dominant frequency from bipolar intracardiac electrograms. IEEE Trans Biomed Eng 2007; 54:165-169.
[4A] Ng J, Kadish A H, Goldberger J J: Effect of electrogram characteristics on the relationship of dominant frequency to atrial activation rate in atrial fibrillation. Heart Rhythm 2006; 3:1295-1305.
[5A] Ng J, Kadish A H, Goldberger J J. Technical considerations for dominant frequency analysis. J Cardiovasc Electrophysiol 2007; 18:757-764.
[6A] Ciaccio E J, Biviano A B, Whang W, Wit A L, Garan H, Coromilas J: New methods for estimating local electrical activation rate during atrial fibrillation. Heart Rhythm 2009; 6:21-32.
[7A] Ciaccio E J, Biviano A B, Whang W, Wit A L, Coromilas J, Garan H. Optimized measurement of activation rate at left atrial sites with complex fractionated electrograms during atrial fibrillation. J Cardiovasc Electrophysiol. 2010; 21:133-143.
[8A] Ciaccio E J, Tennyson C A, Lewis S K, Krishnareddy S, Bhagat G, Green P H. Distinguishing patients with celiac disease by quantitative analysis of videocapsule endoscopy images. Comput Methods Programs Biomed. 2010b; 100:39-48.
[9A] Green P H. Mortality in celiac disease, intestinal inflammation, and gluten sensitivity. JAMA 2009; 302:1225-1226.
[10A] Tennyson C A, Lewis S K, Green P H. New and developing therapies for celiac disease. Therap Adv Gastroenterol. 2009; 2:303-309.
[11A] Rey J F, Repici A, Kuznetsov K, Boyko V, Aabakken L. Optimal preparation for small bowel examinations with video capsule endoscopy. Dig Liver Dis. 2009; 41:486-493.
[12A] Triantafyllou K. Can we improve the diagnostic yield of small bowel video-capsule endoscopy? World J Gastrointest Enclose. 2010; 2:143-146.
[13A] Vilarino, F. Spyridonos, P. Pujol, O. Vitria, J. Radeva, P. de Iorio, F. Automatic Detection of Intestinal Juices in Wireless Capsule Video Endoscopy. Pattern Recognition, 2006. 18th International Conference, Hong Kong; ICPR 2006; 4:719-722.
[14A] Vilarino F, Spyridonos P, Deiorio F, Vitria J, Azpiroz F, Radeva P. Intestinal motility assessment with video capsule endoscopy: automatic annotation of phasic intestinal contractions. IEEE Trans Med Imaging. 2010; 29:246-259.
[15A] Gheorghe C, Jacob R, Bancila I. Olympus capsule endoscopy for small bowel examination. J Gastrointestin Liver Dis, 2007; 16:309-313.
[16A] Press W H, Flannery B P, Teukolsky S A, Vetterling W T, Numerical Recipes in C: The Art of Scientific Computing, 2nd ed. New York, N. Y.: Cambridge Univ. Press, 1992, p501-502.
[17A] Frigo M, Johnson S G. The design and implementation of FFTW3. Proc. IEEE 2005; 93:216-231.
[18A] Ciaccio E J, Tennyson C A, Bhagat G, Lewis S K, Green P H. Classification of videocapsule endoscopy image patterns: comparative analysis between patients with celiac disease and normal individuals. Biomed Eng Online 2010c; 9:44-55.
[19A] Green P H. Celiac disease: how many biopsies for diagnosis? Gastrointest Endosc. 2008; 67:1088-1090.
[20A] Vécsei A, Fuhrmann T, Liedlgruber M, Brunauer L, Payer H, Uhl A. Automated classification of duodenal imagery in celiac disease using evolved Fourier feature vectors. Comput Methods Programs Biomed. 2009 August; 95(2 Suppl):S68-78,
[21A] Charisis, V.; Hadjileontiadis, L. J.; Liatsos, C. N.; Mavrogiannis, C. C.; Sergiadis, G. D. Abnormal pattern detection in Wireless Capsule Endoscopy images using nonlinear analysis in RGB color space. Engineering in Medicine and Biology Society (EMBC), 2010 Annual International Conference of the IEEE Aug. 31 2010-Sep. 4 2010 Buenos Aires. Conf Proc IEEE Eng Med Biol Soc. 2010; 1:3674-7.
[22A] Metzger Y C, Adler S N, shitrit A B, Koslowsky B, Bjarnason I. Comparison of a new PillCam™ SB2 video capsule versus the standard PillCam™ SB for detection of small bowel disease. Reports in Medical Imaging 2009; 2:7-11.
[23A] Culliford A, Daly J, Diamond B, Rubin M, Green P H. The value of wireless capsule endoscopy in patients with complicated celiac disease. Gastrointest Endosc. 2005; 62:55-61.
[24A] Green P H, Rubin M. Capsule endoscopy in celiac disease. Gastrointest Endosc. 2005; 62:797-799.
[25A] Green P H, Rubin M. Capsule endoscopy in celiac disease: diagnosis and management (Review). Gastrointest Endosc Clin N Am. 2006; 16:307-316.
[26A] Seguí S, Igual L, Radeva P, Malagelada C, Azpiroz F Vitria J. A semi-supervised learning method for motility disease diagnostic. Progress in Pattern Recognition, Image Analysis and Applications Lecture Notes in Computer Science, 2007; 4756:773-782.
[27A] Seguí S, Igual L, Vilariño F, Radeva P, Malagelada C, Azpiroz F, Vitrià J. Diagnostic system for intestinal motility disfunctions using video capsule endoscopy. Computer Vision Systems Lecture Notes in Computer Science, 2008; 5008:251-260.

### APPENDIX

The following tested exemplary Fortran code can be useful to compute ensemble average power spectra from multiple CFAEs. The exemplary code can be executed in ˜1 second on a PC-type laptop computer and can be implemented in real time.

```
      parameter (n0=50, n1=500, n2=8192, n3=216,rate=.977)
real en(n1, n1), f(n1), inp(n2, n3) , s(n1, n3)
            do 1 i = 1, n3
                  do 2 j = n1/2+1, n1
                        en(j, 1:j) = 0.
                        do 2 k = 1, n2/j
                              en(j, 1:j) = en(j, 1:j) + inp((k−1)*j+1:(k−1)*j+j, i)
2 continue
            do 3 j = n1/2, n0, −1
                  en(j, 1:j) = en(2*j, 1:j) + en(2*j, j+1:2*j)
3 continue
            do 1 j = n0, n1
                  s(j, i) = sqrt(sum(en(j, 1:j)**2)/n2); if(i.eq.1) f(j) = rate/j
1 continue
en = ensemble vector, rate = digital sampling rate, s = spectral magnitude, f = frequency
inp = input matrix consisting of CFAEs normalized to zero mean and standard deviation = 1.
n0, n1 = range of segment widths (n0, n1) = (50, 500). Frequency f = 977/50 - 977/500 =
19.54Hz - 1.95Hz.
n2 = number of sample points in each CFAE = 8192.
n3 = number of CFAE from which to calculate spectra = 216.
Loop 1 (inner) computes the spectrum s(j, i) based upon Eq. (11), with frequencies given by
f.
Loop 2 zeros the ensemble matrix and computes ensemble averages from (n1)/2 + 1 to n1.
Loop 3 computes ensemble averages from w = n0 to (n1)/2 by averaging the two half
segments from Loop 2 (see exemplary Eq. (21)).
```

1. A method for generating at least one information associated with at least one signal or data received from at least one structure, comprising:
determining at least one basis based on a combination of a plurality of portions of the at least one signal or the data; and
with a computer arrangement, generating the at least one information as a function of the at least one basis.

2-11. (canceled)

12. A non-transitory computer readable medium including instructions thereon that are accessible by a hardware processing arrangement, wherein, when the processing arrangement executes the instructions, the processing arrangement is configured to:
determine at least one basis based on a combination of a plurality of portions of at least one signal or the data; and
generate the at least one information as a function of the at least one basis.

13. The computer readable medium of claim 12, wherein the combination includes at least one of a summation, an average, a weighted average, or a statistical representation.

14. The computer readable medium of claim 12, wherein the summation includes a summation of a plurality of segments of the at least one signal or the data.

15. The computer readable medium of claim 12, wherein the generation of the at least one information comprises applying a transform relating the summation to at least one frequency of the at least one signal so as to generate a power spectrum.

16. The computer readable medium of claim 12, wherein the at least one signal or the data includes at least one of a video-capsule image associated with one of a celiac disease or a cardiac signal as obtained during atrial fibrillation.

17. The computer readable medium of claim 12, wherein the at least one information includes at least one of a dominant frequency, a dominant period, a mean, a standard deviation in a power spectral profile, or a further statistical representation.

18. The computer readable medium of claim 15, further comprising quantifying at least one characteristic associated with the at least one signal or the data based on the transform.

19. The computer readable medium of claim 15, further comprising reducing at least one of a noise, an interference, and an artifact in generating a reconstruction of the at least one signal or the data based on the transform.

20. The computer readable medium of claim 15, further comprising increasing a frequency resolution for a given time period of the at least one signal or the data.

21. The computer readable medium of claim 15, further comprising causing a recognition of a source pattern of the at least one signal or the data based on the transform

22. The computer readable medium of claim 12, wherein the signal or the data is an image.

23. A system for generating at least one information associated with at least one signal or data received from at least one structure, comprising:
a processor which is configured to
i. determine at least one basis based on a combination of a plurality of portions of the at least one signal or the data; and
ii. generate the at least one information as a function of said at least one basis.

24. The system of claim 23, wherein the combination includes at least one of a summation, an average, a weighted average, or a statistical representation.

25. The system of claim 23, wherein the summation includes a summation of a plurality of segments of the at least one signal or the data.

26. The system of claim 23, wherein the generation of the at least one information comprises applying a transform relating the summation to at least one frequency of the at least one signal so as to generate a power spectrum.

27. The system of claim 23, wherein the at least one signal or the data includes at least one of a video-capsule image associated with one of a celiac disease or a cardiac signal as obtained during atrial fibrillation.

28. The system of claim 23, wherein the at least one information includes at least one of a dominant frequency, a dominant period, a mean, a standard deviation in a power spectral profile, or a further statistical representation.

29. The system of claim 26, further comprising quantifying at least one characteristic associated with the at least one signal or the data based on the transform.

30. The system of claim 26, further comprising reducing of at least one of a noise, an interference, or an artifact in generating a reconstruction of the at least one signal or the data based on the transform.

31. The system of claim 26, further comprising increasing a frequency resolution for a given time period of the at least one signal or the data based on the transform.

32. The system of claim 26, further comprising causing a recognition of a source pattern of the at least one signal or the data.

33. The system of claim 23, wherein the signal or the data is an image.
